(19) United States
(12) Patent Application Publication
(10) Pub. No.: US 2024/0427350 A1
(43) Pub. Date: Dec. 26, 2024

(54) POSITIONING SYSTEMS FOR ROBOTIC-SURGERY DEVICES

(71) Applicant: MOMENTIS SURGICAL LTD., Or Yehuda (IL)

(72) Inventors: Idan ROTEM, Tel Aviv (IL); Nelly SOKOL, Tel Aviv (IL); Adam RON, Tel Aviv (IL); Philip MITSEL, Lod (IL)

(21) Appl. No.: 18/685,275

(22) PCT Filed: Aug. 23, 2022
(86) PCT No.: PCT/IB2022/057894

§ 371 Date: Feb. 21, 2024

Related U.S. Application Data
(60) Provisional application No. 63/235,832, filed on Aug. 23, 2021.

(51) Int. Cl. G05D 1/661; A61B 34/37; A61B 90/30

(52) U.S. Cl. CPC G05D 1/661; A61B 34/37; A61B 90/30

(57) ABSTRACT

A positioning system for a robotic-surgery device comprises a lower portion comprising a pillar section extending from a wheeled base, an upper portion supported by the pillar section and comprising a docking interface adapted for securing thereto a motor-control unit of a robotic-surgery device, and electronic circuitry programmed to cause the upper portion to simultaneously displace vertically and pitch about a pitch-axis member mediating between the pillar section and the upper-portion, in response to a remote user input, so as to pivot a robotic-surgery device secured to the docking interface about a distal end of a surgical arm proximally seated in a surgical-arm-receiving volume of the motor-control unit.

### CROSS-REFERENCE TO RELATED APPLICATIONS

This patent application claims the benefit of U.S. Provisional Patent Application No. 63/235,832, filed on Aug. 23, 2021, which is incorporated herein by reference in its entirety.

### FIELD OF THE INVENTION

The present invention relates to positioning systems include carts and mobile pedestals for use with robotic-surgery devices and particularly to robotic-surgery devices comprising one or more elongate mechanical arms.

### BACKGROUND

It is well established that there are benefits of minimally invasive surgery. Instruments for such surgery typically have a surgical end effector located at the distal end of an articulated surgical arm (preferably with minimum diameter) that is inserted through a small opening (e.g., body wall incision, natural orifice) to reach a surgical site. In some instances, surgical instruments can be passed through a cannula and an endoscope can be used to provide images of the surgical site.

Surgical instruments have been developed that utilize an end effector (e.g., a surgical tool such as for tissue fusing or cutting, or a measurement tool) for convenience, accuracy, and wellbeing of the subject. In some cases, articulated surgical arms have one or more bending portions which are controlled remotely using various input devices (e.g., hand and foot controls) to ultimately control the location of the end effector and change its orientation with reference to the surgical arm's longitudinal axis.

Motor-control units include gears which mesh with gears of surgical arms for precise control of the arms. Positioning systems in the form of wheeled and unwheeled carts, mobile pedestals and the like can be used to support and interface with he motor-control units and bring the surgical instruments in proximity to target locations for initiating surgical procedures. Existing positioning systems are known to lack mechanisms for precise adjustment of positions, for accurately aligning surgical arms with target vectors for initiating surgery, and for moving and orienting surgical instruments through three-dimensional space for achieving the necessary accurate alignment.

### SUMMARY OF THE INVENTION

According to embodiments disclosed herein, a positioning system for a robotic-surgery device comprises: (a) a lower portion comprising a pillar section extending from a wheeled base; (b) an upper portion supported by the pillar section and comprising a docking interface adapted for securing thereto a motor-control unit of a robotic-surgery device, the upper portion configured for displacing vertically in response to a first remote user input and for pitching, in response to a second remote user input, about a pitch-axis member mediating between the pillar section and the upper-portion; and (c) electronic circuitry programmed to cause the upper portion to simultaneously displace vertically and pitch about the pitch-axis member, in response to a third remote user input, so as to pivot a robotic-surgery device secured to the docking interface about a distal end of a surgical arm proximally seated in a surgical-arm-receiving volume of the motor-control unit.

In some embodiments, the electronic circuitry can be programmed to effect the pivoting of the robotic-surgery device about the distal end of the surgical arm without displacing the distal end of the surgical arm.

In some embodiments, the distal end of the surgical arm can be characterized by having an end-effector coupled thereto, and the pivoting of the robotic-surgery device about the distal end of the surgical arm includes pivoting the robotic-surgery device about a distal end of the end-effector.

In some embodiments, the positioning system can additionally comprise a user-input device arranged to receive the first, second and third remote user inputs from a user. In some embodiments, the docking interface can be configured for displacing longitudinally in response to a fourth remote user input. In some embodiments, the user-input device can additionally be arranged to receive the fourth remote user input from the user.

In some embodiments, the electronic circuitry can additionally be programmed to cause the docking interface to displace longitudinally simultaneously with the vertical displacing and pitching of the upper portion, in response to a fifth remote user input.

In some embodiments, the positioning system can additionally comprise a visual aid for aligning the surgical arm with a target vector. In some embodiments, the target vector can describe at least one of a position of a surgical access channel and an orientation of a surgical access channel.

In some embodiments, the pillar section can be at least partly telescopic.

A method is disclosed, according to embodiments; the method comprises: (a) providing the positioning system of any preceding claim in an assembled state in which the motor-control unit is secured to the docking interface and a surgical arm is proximally seated in a surgical-arm-receiving volume of the motor-control unit; (b) positioning the positioning system in proximity to a target location; (c) receiving the third remote user input when the distal end of the surgical arm is disposed at the target location; and (d) in response to receiving the third remote user input, simultaneously displacing the upper portion vertically and pitch the upper portion about the pitch-axis member, so as to pivot the robotic-surgery device about the distal end of the surgical arm.

In some embodiments, the method can additionally comprise, after the positioning: vertically displacing the upper portion until the distal end of the surgical arm is disposed at the target location.

In some embodiments, the target location can be proximate to a surgical access channel.

In some embodiments, the method can additionally comprise: ceasing the pivoting of the robotic-surgery device about the distal end of the surgical arm when the surgical arm is oriented to be aligned with the surgical access channel. In some embodiments, it can be that the ceasing is in response to an indication of a visual aid that the surgical arm is oriented to be aligned with the surgical access channel. In some embodiments, the visual aid can include a laser illuminator.

According to embodiments disclosed herein, a positioning system for a robotic-surgery device comprises: (a) an upper portion comprising a docking interface adapted for securing thereto a motor-control unit of a robotic-surgery device; and (b) a lower portion comprising: (i) respective pluralities of support elements arranged to support the positioning system in respective positioning, position-adjusting, and stationary operating modes, the respective pluralities comprising respective pluralities of wheels, ball casters, and wheelless legs, and (ii) one or more mode-change pedals configured to transfer support of the positioning system from one respective plurality of support elements to another, the mode-change pedals arranged such that (A) an application of a mode-change pedal when the positioning system is supported by the plurality of wheels is effective to transfer support of the positioning system to the plurality of ball casters, and (B) an application of a mode-change pedal when the positioning system is supported by the plurality of ball casters is effective to transfer support of the positioning system to the plurality of wheelless legs.

In some embodiments, at least one of the one or more mode-change pedals can be arranged to lift the plurality of wheels when applied.

In some embodiments, it can be that the one or more mode-change pedals comprises a first mode-change pedal and a second mode-change pedal, and when the positioning system is supported by the plurality of wheels, an application of the first mode-change pedal is effective to transfer support of the positioning system to the plurality of ball casters, and an application of the second mode-change pedal is effective to transfer support of the positioning system to the plurality of wheelless legs.

In some embodiments, it can be that each wheel of the plurality of wheels has a first diameter and each ball caster of the plurality of ball casters has a second diameter, the first diameter being at least three times greater than the second diameter.

In some embodiments, the upper portion can be configured for displacing vertically in response to a remote user input.

In some embodiments, the upper portion can be configured for pitching, in response to a remote user input, about a pitch-axis member mediating between the lower portion and the upper-portion.

In some embodiments, the docking interface can be configured for displacing longitudinally in response to a remote user input.

A method is disclosed according to embodiments; the method comprises: (a) providing a positioning system for a robotic-surgery device, the positioning system comprising (i) respective pluralities of support elements arranged to support the positioning system in respective positioning, position-adjusting, and stationary operating modes, the respective pluralities comprising respective pluralities of wheels, ball casters, and wheelless legs, and (ii) one or more mode-change pedals configured to transfer support of the positioning system from one respective plurality of support elements to another; (b) positioning the positioning system, with the positioning system supported by the plurality of wheels; and (c) applying one of the one or more mode-change pedals to transfer support of the positioning system to the plurality of wheelless legs, thereby placing the positioning system in the stationary operating mode.

In some embodiments, the method can additionally comprise, after the positioning of the positioning system: (i) applying one of the one or more mode-change pedals to transfer support of the positioning system from the plurality of wheels to the plurality of ball casters; and (ii) adjusting the positioning of the positioning system with the positioning system supported by the ball casters.

In some embodiments, at least one of the one or more mode-change pedals can be arranged to lift the plurality of wheels when applied.

According to embodiments disclosed herein, medical apparatus comprises: (a) a positioning system comprising a docking interface adapted for securing thereto a motor-control unit of a robotic-surgery, the motor-control unit comprising one or more surgical-arm-receiving volumes; (b) an illumination source coupled to the docking interface and operable to generate illumination in a positioning-system-distal direction, the illumination source being offset from the one or more surgical-arm-receiving volumes at least in a transverse direction that is orthogonal to the positioning-system-distal direction; and (c) an access-channel-assembly frame (i) comprising first and second iron sights aligned with each other to define an illumination target and an iron-sight-distal direction, and (ii) configured to have coupled thereto a surgical access channel shaped to allow traversal of respective distal portions of one or more surgical arms of the robotic-surgery device, the coupling being such that the first and second iron sights are offset from the coupled surgical access channel at least in a transverse direction that is orthogonal to the iron-sight-distal direction, wherein a transverse-direction offset of the illumination source from the one or more arm-receiving volumes is substantially the same as a transverse-direction offset of the first and second iron sights from the access channel.

In some embodiments, it can be that when (i) the access-channel-assembly frame has a surgical access channel coupled thereto and is disposed at a surgical entry point, and/or (ii) the docking interface is positioned in a proximate-to-surgical-entry location in an assembled state in which the motor-control unit is mounted thereto, and one or more surgical arms are proximally received in respective arm-receiving volumes of the motor-control unit, and/or (iii) the illumination source is operated to generate illumination in the positioning-system-distal direction, adjusting at least one of a position of the docking interface and an orientation of the docking interface, such that the illumination source illuminates the illumination target, is effective to align the one or more surgical arms for passage of distal ends thereof through the surgical access channel. In some embodiments, the illumination source can include a laser illuminator.

In some embodiments, the iron sights can be fold-out sights, and the defined illumination target and iron-sight-distal direction are defined with the sights folded-out.

In some embodiments, the docking interface can be configured to be displaced vertically in response to a remote user input.

In some embodiments, the docking interface can be configured to be pivoted, in response to a remote user input, about a pitch-axis member of the positioning system.

In some embodiments, the docking interface can be configured for displacing longitudinally in response to a remote user input.

A method is disclosed, according to embodiments, of employing the medical apparatus according to any one of the embodiments disclosed hereinabove. The method comprises: (a) placing, at a surgical entry point, the surgical access channel, coupled to the access-channel-assembly frame; (b) positioning, in a proximate-to-surgical-entry location, the positioning system, in an assembled state in which the motor-control unit is mounted thereto, and the one or more surgical arms are proximally received in respective arm-receiving volumes of the motor-control unit; (c) operating the illumination source to generate illumination in the positioning-system-distal direction; and (d) adjusting at least one of a position of the docking interface and an orientation of the docking interface so that the illumination source illuminates the illumination target.

In some embodiments, the adjusting can be effective to align the one or more surgical arms for passage of distal ends thereof through the surgical access channel.

According to embodiments disclosed herein, a positioning system for a robotic-surgery device comprises: (a) a lower portion comprising a pillar section extending from a wheeled base; (b) an upper portion supported by the pillar section and comprising a docking interface adapted for securing thereupon a motor-control unit of a robotic-surgery device, the upper portion configured for displacing vertically and for pitching about a pitch-axis member mediating between the pillar section and the upper-portion; and (c) electronic circuitry programmed to cause the upper portion to simultaneously displace vertically and pitch about the pitch-axis member to pivot the motor-control unit about a remote target point to align an operative vector of the motor-control unit with a target vector at the remote target point.

In some embodiments, the aligning of the operative vector with the target vector can include: (i) lining up the operative vector of the motor-control unit to intercept the remote target point, and/or (ii) pivoting the motor-control unit about the remote target point to align the operative vector of the motor-control unit with a target vector at the remote target point. In some embodiments, the aligning of the operative vector with the target vector can include aligning an offset vector with an offset target vector.

In some embodiments, the electronic circuitry can be programmed to cause the upper portion to simultaneously displace vertically and pitch about the pitch-axis member while the motor-control unit is secured to the docking interface.

In some embodiments, the electronic circuitry can be programmed to cause the upper portion to simultaneously displace vertically and pitch about the pitch-axis member while a surgical arm is proximally seated in a surgical-arm-receiving volume of the motor-control unit.

In some embodiments, a surgical-arm-receiving volume of the motor-control unit can be configured to receive a proximal portion of a surgical arm.

In some embodiments, a surgical arm proximally seated in the surgical-arm-receiving volume can be aligned with the operative vector.

In some embodiments, the electronic circuitry can be additionally programmed to cause the docking interface to displace longitudinally simultaneously with the vertical displacing and pitching of the upper portion.

In some embodiments, the electronic circuitry can be additionally programmed to cause the docking interface to displace longitudinally after the aligning of the operative vector with the target vector.

In some embodiments, the positioning system can additionally comprise a visual aid for aligning the operative vector with the target vector.

In any of the embodiments of position systems disclosed herein, the positioning system can be configured to provide electrical power to the motor-control unit.

### BRIEF DESCRIPTION OF THE DRAWINGS

The invention will now be described further, by way of example, with reference to the accompanying drawings, in which the dimensions of components and features shown in the figures are chosen for convenience and clarity of presentation and not necessarily to scale. In the drawings:

FIGS. 1A and 1B are, respectively, schematic elevation and perspective views of a positioning system according to embodiments of the present invention.

FIG. 2 is a schematic elevation view of a positioning system in an assembled state together with a robotic-surgery device comprising a surgical arm, according to embodiments of the present invention.

FIG. 3 shows the assembled-state positioning system of FIG. 2 where the upper portion of the positioning system is vertically displaced, according to embodiments of the present invention.

FIGS. 4A and 4B show the assembled-state positioning system of FIG. 2 where the upper portion of the positioning system is pivoted about a pitch axis, according to embodiments of the present invention.

FIG. 5 shows the assembled-state positioning system of FIG. 2 where a docking interface of the upper portion of the positioning system is displaced distally, according to embodiments of the present invention.

FIGS. 6A and 6B are respective schematic elevation and perspective views of a positioning system comprising handles, pedals and a control panel, according to embodiments of the present invention.

FIG. 7 is a schematic view of a control panel of a positioning system, according to embodiments of the present invention.

FIG. 8 is a schematic view of mode-changing pedals of a positioning system, according to embodiments of the present invention.

FIG. 9 is a schematic elevation view of the positioning system of FIGS. 6A-B, where the upper portion of the positioning system is both vertically displaced and pivoted about a pitch axis, according to embodiments of the present invention.

FIG. 10 is a schematic elevation view of the positioning system of FIGS. 6A-B, where the upper portion of the positioning system is vertically displaced, and a docking interface of the upper portion is displaced distally, according to embodiments of the present invention.

FIG. 11 is a schematic elevation view of the positioning system of FIGS. 6A-B, where the upper portion of the positioning system is pivoted about a pitch axis, and a docking interface of the upper portion is displaced distally, according to embodiments of the present invention.

FIG. 12 is a schematic elevation view of the positioning system of FIGS. 6A-B, where the upper portion of the positioning system is both vertically displaced and pivoted about a pitch axis, and a docking interface of the upper portion is displaced distally, according to embodiments of the present invention.

FIG. 13A is a schematic elevation view of the positioning system of FIG. 9 in an assembled state, showing an angle between an orientation of a target vector and the surgical arm of the robotic-surgery device, according to embodiments of the present invention.

FIG. 13B shows the assembled-state positioning system of FIG. 13A aligned with the target vector, according to embodiments of the present invention.

FIG. 14 schematically shows the assembled-state positioning system of FIG. 12 in an assembled state, where the upper portion of the positioning system is vertically displacing and pivoting about a pitch axis, and a docking interface of the upper portion is displacing distally, according to embodiments of the present invention.

FIG. 15 shows a detail of a lower portion of a positioning system showing a wheel and a wheelless leg, according to embodiments of the present invention.

FIG. 16 shows a detail of a lower portion of a positioning system showing a ball caster and a wheelless leg, according to embodiments of the present invention.

FIG. 17 shows a detail of a lower portion of a positioning system showing application of a mode-change pedal, according to embodiments of the present invention.

FIG. 18 is a schematic perspective view of an upper portion of a positioning system, showing an illumination source arranged to generate illumination in a positioning-system-distal direction, according to embodiments of the present invention.

FIG. 19A shows access-channel-assembly frame comprising fold-out iron sights and coupled to an access channel, according to embodiments of the present invention.

FIG. 19B shows a detail of the frame of FIG. 19A with the iron sights aligned to define an illumination target according to embodiments of the present invention.

FIG. 20 is a schematic, partial distal-end elevation view of an assembled-state positioning system and an access-channel-assembly frame with iron sights and access channel, according to embodiments of the present invention.

FIG. 21 shows an access-channel-assembly frame comprising fold-out iron sights and coupled to an access channel, in a folded state, according to embodiments of the present invention.

FIGS. 22A, 22B, 22C, 23A and 23B show flowcharts of methods and method steps according to embodiments of the present invention.

FIG. 24 shows a flowchart of a method of employing a medical apparatus according to embodiments of the present invention.

FIG. 25A is a schematic elevation view of a positioning system, showing an angle between an orientation of an operative vector and a target vector, according to embodiments of the present invention.

FIG. 25B shows the positioning system of FIG. 25A when the operative vector is aligned with the target vector, according to embodiments of the present invention.

FIG. 25C schematically shows the positioning system of FIGS. 25A-B, where the upper portion of the positioning system is vertically displacing and pivoting about a pitch axis, and a docking interface of the upper portion is displacing distally, according to embodiments of the present invention.

FIG. 26A is a schematic elevation view of a positioning system, showing an angle between an orientation of an operative vector of the motor-control unit of a robotic-surgery device and a target vector, according to embodiments of the present invention.

FIG. 26B shows the positioning system of FIG. 26A when the operative vector is aligned with the target vector, according to embodiments of the present invention.

FIG. 26C schematically shows the positioning system of FIGS. 26A-B, where the upper portion of the positioning system is vertically displacing and pivoting about a pitch axis, and a docking interface of the upper portion is displacing distally, with the motor-control unit of the robotic-surgery device present, according to embodiments of the present invention.

### DETAILED DESCRIPTION OF EMBODIMENTS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. Throughout the drawings, like-referenced characters are generally used to designate like elements.

Throughout this disclosure, subscripted reference numbers (e.g., 10₁ or 10_{A}) may be used to designate multiple separate appearances of elements of a single species, whether in a drawing or not; for example: 10₁ is a single appearance (out of a plurality of appearances) of element 10. The same elements can alternatively be referred to without subscript (e.g., 10 and not 10₁) when not referring to a specific one of the multiple separate appearances, i.e., to the species in general.

The descriptive terms ‘clockwise’ and ‘counterclockwise’ when used in this disclosure are to be understood only with reference to orientations of the positioning system and its components as illustrated in the various figures. The terms are merely used for convenience and describe only a specific respective viewing perspective of a given figure. A ‘clockwise’ movement when viewed from one side of the positioning system, i.e., the viewing perspective of a given figure, would be counterclockwise if viewed from the other side of the positioning system, and vice versa. ‘Forward’ and ‘reverse’ with respect to a pitch movement mean the same as ‘clockwise’ and ‘counterclockwise’ as specifically illustrated in the various figures, in which the distal direction (towards a patient) is to the right of the respective figures.

Embodiments disclosed herein relate to positioning systems for robotic-surgery devices using one or more surgical mechanical arms, i.e., articulated mechanical arms, using a plurality of different operating modes and/or a plurality of different input devices.

Whenever ‘arm’ is used herein or in the appended claims, it means an articulated mechanical arm that is part of a surgical system or electrosurgical system and used for performing or helping to perform surgical (including, without limitation electrosurgical and imaging) actions inside a human subject's body. An articulated arm can also be considered flexible. An arm may include an end effector, which is used herein to mean a tool or device used in connection with surgery, electrosurgery, diagnosis or imaging when deployed within a human body. An end effector may be supplied as part of an arm, i.e., already mounted, mechanical attached and/or integrated with the power and communications conveyances of the arm; in some embodiments an arm an end effector may be provided separately for assembly and/or integration into a working unit before or even during a surgical operation, i.e., before insertion into a subject's body. A ‘robotic-surgery device’ as used herein means a device having one or more surgical mechanical arms and a motor unit or motor-control unit for housing and controlling the one or more arms.

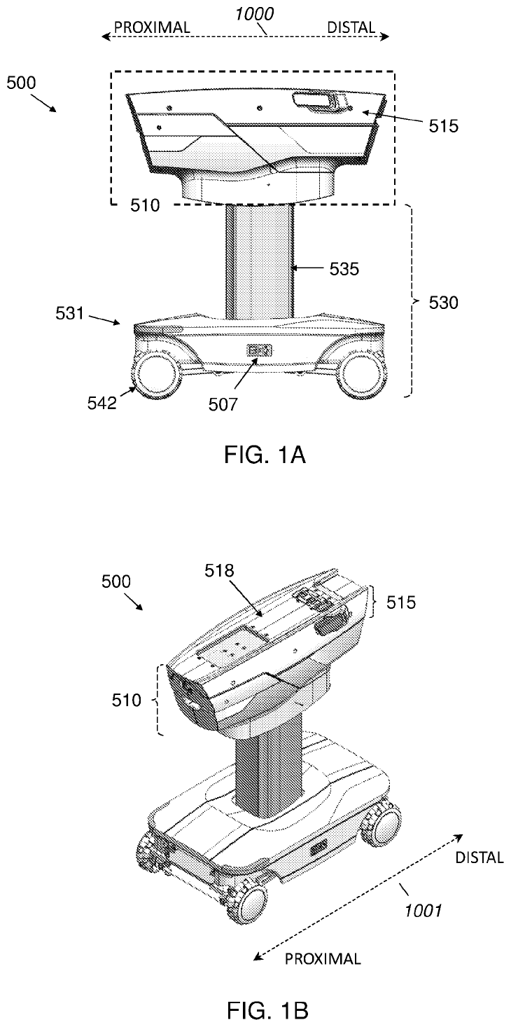

Referring to the figures and in particular to FIGS. 1A and 1B, a positioning system 500 according to embodiments is illustrated in side-elevation and proximal-perspective views. Respective arrows 1000 and 1001 of FIGS. 1A and 1B indicate proximal and distal directions as the terms are used throughout this disclosure. ‘Distal’ means the direction closer to, or in the direction of, a patient, while ‘proximal’ means the direction farther from, or away from, the patient. The exemplary positioning system 500 of FIGS. 1A and 1B comprises a lower portion 530, which includes a pillar 535 supported by a wheeled base 531. The wheeled base 531 includes a plurality of wheels 542 pivotably mounted thereto such that the positioning system 500 is supported by the plurality of wheels 542. The term ‘supported by’ is used throughout the disclosure and in the claims appended thereto to mean that most, e.g., at least 70% or at least 80%, or at least 90%, or all, of the weight of the former (positioning system 500) is supported by the latter (plurality of wheels 542). The positioning system 500 further comprises an upper portion 510 which includes a slidable docking interface 515, an upwards-facing exposed surface of which includes docking arrangements 518 for securing thereto a motor-control unit of a robotic-surgery device 100 (not shown in FIGS. 1A-B). As shown in FIG. 1A, the wheeled base 531—or any other suitable portion of the positioning system 500 in accordance with specific designs-can include a power interface 507 for receiving electrical power from an external source and/or for charging an onboard power source, e.g., a battery (not shown) installed in the positioning system 500, e.g., in the wheeled base 531. The position of power interface 507 is merely illustrative and in some implementations of the embodiments can be placed on a proximal surface or any other surface of the positioning system 500, e.g., in accordance with operating room design or with safety requirements. In embodiments, an on-board power system fed by power interface 507 and or by the aforementioned battery can be utilized to power a robotic-surgery device 100. According to such embodiments, the power can be supplied to the robotic-surgery device 100 through the docking arrangements 518 or through an electrical wire, e.g., a wire extending proximally from the robotic-surgery device 100 comprising an electrical plug compatible with an electrical socket provided on the positioning system 500. The electrical socket can be part of power interface 507 or elsewhere on the positioning system 500. In another example, an electrical connection for the robotic-surgery device 100 can be extended, when needed, from a compartment within the base 531 or any other portion of the positioning system 500. Providing power to the robotic-surgery device from the positioning system simplifies set-up for a surgical operation by requiring connection of a single electrical plug in order to enable the powered operation of both the positioning system 500 and the robotic-surgery device 100. The powered positioning system 500 thus is configured to act as power supply and/or a charging station for the robotic-surgery device 100. In some applications, when the powered positioning system 500 is plugged into an electrical outlet the robotic-surgery device 100 is enabled, and when the positioning system 500 is not plugged in, the robotic-surgery device 100 is disabled. Additionally or alternatively, in some applications, when the electrical systems of the positioning system 500 is power up (electricity is turned on), e.g., by an on/off switch (not shown), the robotic-surgery device 100 is powered up, and when the positioning system 500 is powered down (electricity is turned off), e.g., by the on/off switch, the robotic-surgery device 100 is also powered down. Similarly, in some applications, an emergency stop of the positioning system 500 is propagated as an emergency stop of the robotic-surgery device 100 as well. In some applications, when electrical power is available to the robotic-surgery device 100 from the positioning system 500, the robotic-surgery device 100 is thereby enabled to be turned on and off directly, as well as from the positioning system 500 by any one of: cutting power to the robotic-surgery device 100 (whether as an emergency stop or not), shutting off the power of the positioning system 500 (whether as an emergency stop or not), or disconnecting the positioning system 500 from the electricity.

The wheeled base 531—or any other suitable portion of the positioning system 500 in accordance with specific designs—can include one or more motors and/or other mechanical actuators for effecting movements of portions of the positioning system 500, and/or of the positioning system 500 itself, using electric power from the external source or from the onboard power source.

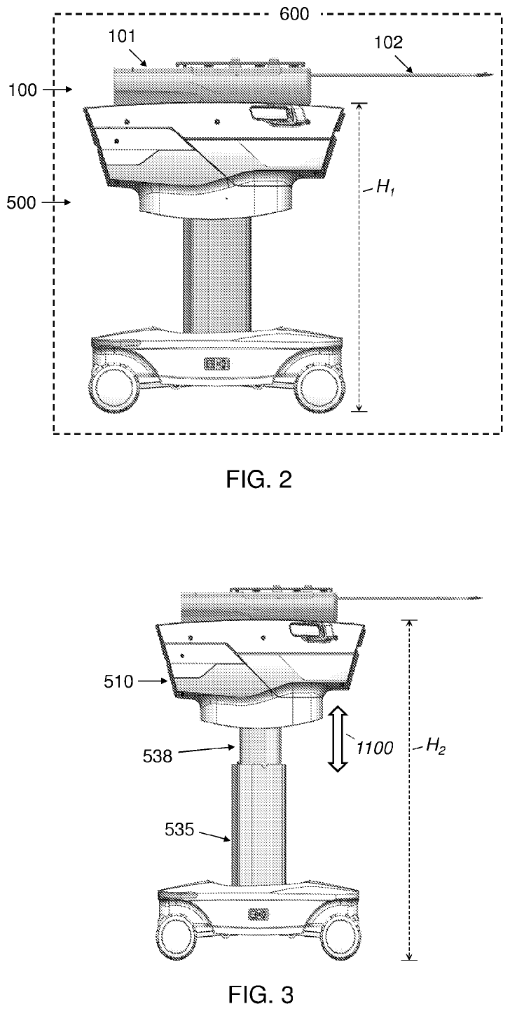

FIG. 2 schematically illustrates an assembled-state positioning system 600. The assembled state is a state in which a robotic-surgery device 100 is installed on the positioning system 500 and one or more surgical arms 102 are proximally seated in respective arm-receiving volumes of a motor-control unit 101 of the robotic-surgery device 100. The exemplary robotic-surgery device 100 of FIG. 2 comprises a motor-control unit 101 housing motors and gears for controlling one or more surgical arms 102. Each one of the one or more surgical arms 102 is proximally seated, i.e., a proximal portion of each arm 102 is seated, in an arm-receiving volume (not shown) of the motor-control unit 101, as to be secured therein and to be controlled thereby, e.g., by having gears of the surgical arm 102) mesh with corresponding gears in the respective arm-receiving volume(s). The height of an exemplary positioning system 500, as indicated by the arrow marked H₁ in FIG. 2, is between 50 and 150 cm (all ‘between’ ranges in this disclosure are inclusive), or between 50 and 70 cm, or between 50 and 90 cm, or between 50 and 120 cm, or between 50 and 150 cm, or between 70 and 90 cm, or between 70 and 120 cm, or between 70 and 150 cm, or between 90 and 120 cm, or between 90 and 150 cm, or between 120 and 150 cm.

We now refer to FIGS. 3, 4A, 4B and 5.

In embodiments, it can be desirable for a positioning system 500 such as those disclosed herein to be non-monolithic, but rather to comprise moving parts that can be moved in various directions, e.g., for facilitating the aligning of surgical arms with target vectors associated with patient-proximate surgical arrangements such as surgical access channels.

FIG. 3 shows a an assembled-state positioning system 600, e.g., similar to the assembled-state positioning system 600 of FIG. 2 and comprising an upper portion 510 configured to displace vertically. The bidirectional displacing capability of the upper portion 510 is indicated by arrow 1100. The pillar section 535 of FIG. 3 is telescopic, as evidenced by telescopic section 538 rising from within the pillar section 535. In other exemplary designs, the pillar section 535 is not necessarily telescopic, and a piston is provided, e.g., within the pillar 535, for raising and lowering the upper portion 510. The displacing is typically a powered displacing, for example, effected by an onboard motor or other actuator and powered by an external or onboard power source. The vertical displacing can be initiated in response to a user input such as a remote user input received by a user-input device, e.g., user-input panel 580 of FIG. 7, that can be attached to the positioning system 500 or disposed elsewhere. The fully-extended height of an exemplary positioning system 500 wherein the upper portion is vertically displaced to a maximum extent, as indicated by the arrow marked H₂ in FIG. 3, is between 100 and 200 cm (all ‘between’ ranges in this disclosure are inclusive), or between 100 and 120 cm, or between 100 and 150 cm, or between 100 and 180 cm, or between 120 and 150 cm, or between 120 and 180 cm, or between 120 and 200 cm, or between 150 and 180 cm, or between 150 and 200 cm. The total extension of the height, i.e., the difference between height H₁ and height H₂, is between 10 and 100 cm, or between 10 and 30 cm, or between 10 and 50 cm, or between 10 and 75 cm, or between 30 and 50 cm, or between 30 and 75 cm, or between 30 and 100 cm, or between 50 and 75 cm, or between 50 and 100 cm, or between 75 and 100 cm, or more.

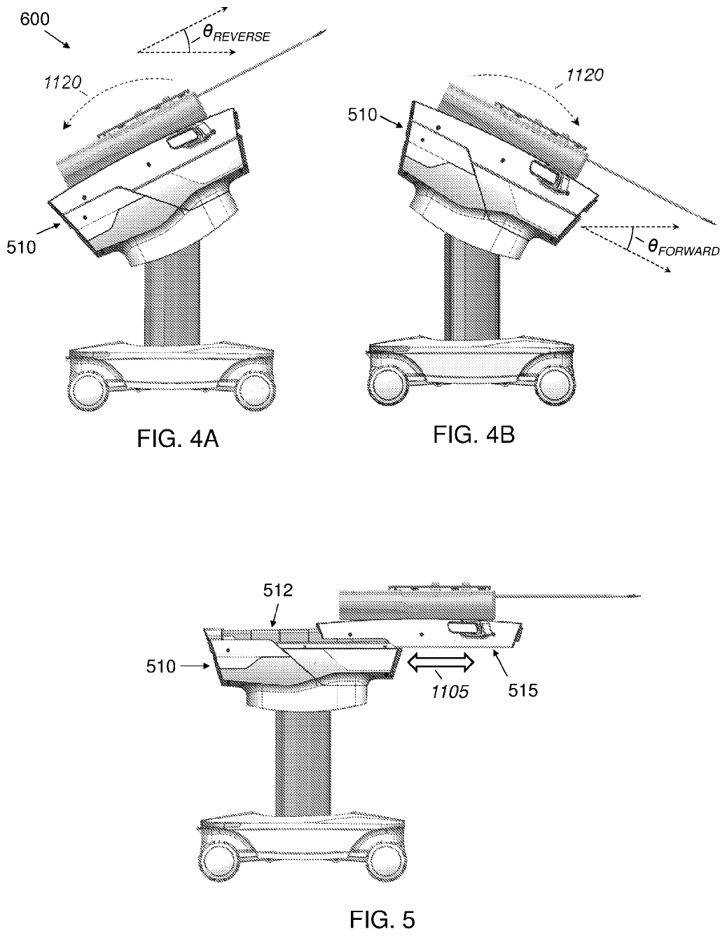

FIGS. 4A and 4B show an assembled-state positioning system 600, e.g., similar to the assembled-state positioning system 600 of FIG. 2 and comprising an upper portion 510 configured to pivot about a pitch-axis member (not shown) that mediates between the pillar section 535 and the upper portion 510. The bidirectional pitching of the upper portion 510 is indicated by respective arrows 1120 in both FIGS. 4A and 4B. The pitching is typically a powered pivoting, for example, effected by an onboard motor or other actuator and powered by an external or onboard power source. The pivoting motion can be imparted by any suitable mechanical arrangement such as gears, pistons and/or clutches installed in the positioning system 500 and in communication with the pivot-axis member. As illustrated in FIG. 4A, the upper portion 510 can be configured for counterclockwise pitching (i.e., counterclockwise from the perspective of FIG. 4A) to a maximum pivot angle of θ_{REVERSE}. In embodiments, the pivot angle can correspond to a patient orientation during surgery, such as, e.g., a reverse Trendelenburg position. In embodiments, maximum pivot angle θ_{REVERSE} is at least 10°, or at least 15°, or at least 20°, or at least 25°, or at least 30°, or at least 35°, or at least 40°, or at least 45°. As illustrated in FIG. 4B, the upper portion 510 can be configured for clockwise pitching (i.e., clockwise from the perspective of FIG. 4B) up to a maximum pivot angle of θ_{FORWARD}. In embodiments, maximum pivot angle θ_{FORWARD} is at least 10°, or at least 15°, or at least 20°, or at least 25°, or at least 30°, or at least 35°, or at least 40°, or at least 45°. In embodiments, the pivot angle can correspond to a patient orientation during surgery, such as, e.g., a Trendelenburg position. In some embodiments, θ_{REVERSE} and θ_{FORWARD} are equal, and in other embodiments, based upon specific design choices for the cart 500, they are not.

FIG. 5 shows an assembled-state positioning system 600, e.g., similar to the assembled-state positioning system 600 of FIG. 2, where the upper portion 510 comprises a slidable docking interface 515 configured to displace longitudinally, i.e., distally from an initial position of the upper portion 510. The term ‘longitudinal’ (or ‘longitudinally’) generally means in either or both of the two directions represented by the proximal-distal axis indicated in FIGS. 1A and B by respective arrows 1000 and 1001. The bidirectional displacing of the docking interface 515 is indicated by arrow 1105 in FIG. 5. The longitudinal displacing is typically a powered displacing, for example, effected by an onboard motor or other actuator and powered by an external or onboard power source. The displacing motion can be imparted by any suitable mechanical arrangement such as a piston, or, in the non-limiting example of FIG. 5, a telescoping member 512. The slidable docking interface 515 can be configured to displace distally by up to 120 cm, or up to 100 cm, or up to 80 cm, or up to 60 cm, or up to 40 cm.

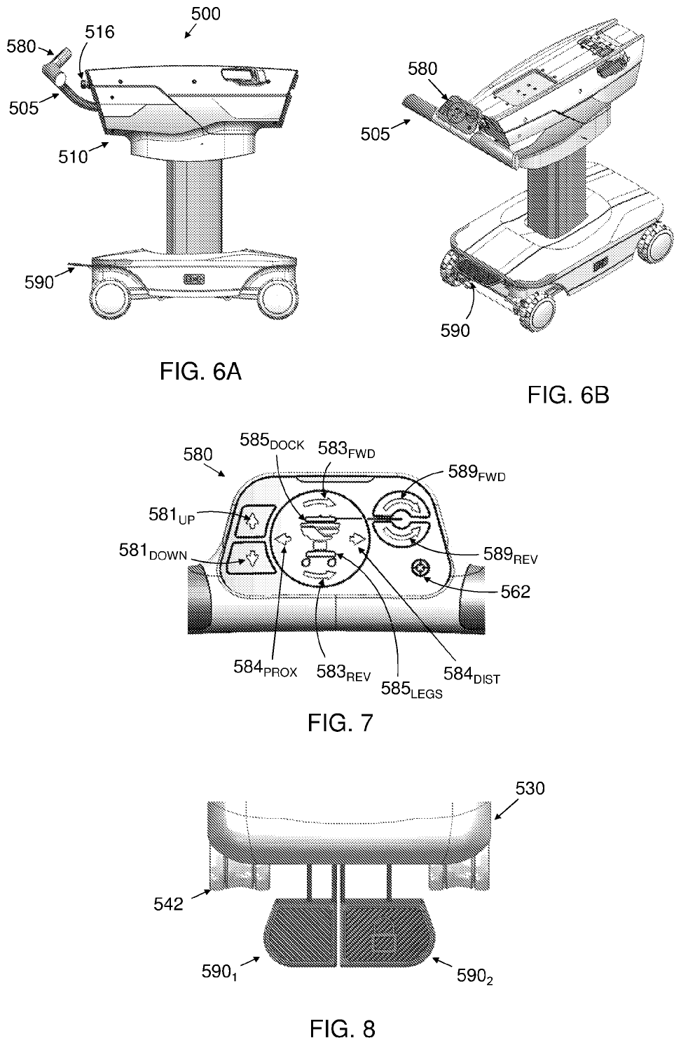

A positioning system 500 according to embodiments is schematically illustrated in respective side-elevation and proximal-perspective views in FIGS. 6A and 6B. The positioning system 500 includes a handle member 505 which can include one or handles for use in pulling and/or pushing the positioning system 500 in any direction in which it is designed to move. In some embodiments this can include distal-proximal motion only, and in other embodiments it can include motion in any direction, for example for adjusting or fine-tuning a position attained with the distal-proximal motion. In the non-limiting example of FIGS. 6A and 6B, the handle member 505 is used as an attachment point for a user-input panel 580. A user-input panel 580 can be used for controlling various functions of the positioning system 500, including, without limitation, the displacing and pitching of components discussed hereinabove with reference to FIGS. 3, 4A, 4B and 5. While this placement of the user-input panel 580 has ergonomic advantages in that the user-input panel 580 is conveniently located for a user standing proximally behind the positioning system 500, e.g., for pushing or pulling it, the user-input panel 580 can be disposed anywhere on the positioning system 500 and can optionally be attachably detachable, e.g., dockable. Other input devices, such as a motion-stop button 516 for ceasing all displacing and pivoting of positioning-system components, can be installed or attached to the positioning system 500.

Referring now to FIG. 7, details of an exemplary user-input device (or, equivalently, user-input panel or control panel) 580 are shown. A user-input panel can include any or all of the examples of controls discussed in this paragraph, and can include other examples of controls not specifically discussed in this paragraph, which can perform additional or alternative functions and/or combine functions discussed in this paragraph. Further, the controls can be laid out in any suitable arrangement, and the layout and graphic design shown in FIG. 7 are to illustrate a particularly ergonomic example. As shown in FIG. 7, respective up and down vertical-displacement controls 581_{UP}, 581_{DOWN} are provided for controlling (e.g., initiating, ceasing, etc.) vertical displacement of the upper portion 510, as illustrated in FIG. 3, in response to a user input received by (or, equivalently for all user inputs, at) the user-input panel 580. Respective clockwise and counterclockwise pitch controls 583_{FWD}, 583_{REV} are provided for controlling forward and reverse pitch-axis pivoting, respectively of the upper portion 510, as illustrated in FIGS. 4A and 4B, in response to a user input received by the user-input panel 580. To generalize from the terms ‘forward’ and ‘reverse’ as used here with respect to pitching, a forward pitching movement is the pivoting movement that raises the proximal end of the upper portion 510 and lowers the distal end, while the reverse pitching movement is the pivoting movement that lowers the raises the proximal end of the upper portion 510 and raises the distal end. Respective proximal and distal longitudinal-displacement controls 584_{PROX}, 584_{DIST} are provided for controlling displacement of the slidable docking interface 515, in response to a user input received by the user-input panel 580. Notification elements 585, e.g., lights, light-emitting diodes (LEDs), liquid-crystal displays (LCDs) or the like, are provided for displaying a status of the positioning system 500 or of any one or more of its components. A first notification element 585_{LEGS} on the control panel 580 of FIG. 7 is illuminated when the wheelless legs 545 of the positioning system 500 are deployed, e.g., when the wheels 542 are raised or when the wheelless legs 545 are lowered. A second notification element 585_{DOCK} on the control panel 580 of FIG. 7 is illuminated when a motor-control unit 101 of a robotic-surgery device 100 is secured, e.g., docked and locked, to the slidable docking interface 515 of the positioning system 500.

In another example of a control panel (not illustrated), some or all of the controls and notification elements reside in an interactive user interface such as a touchscreen.

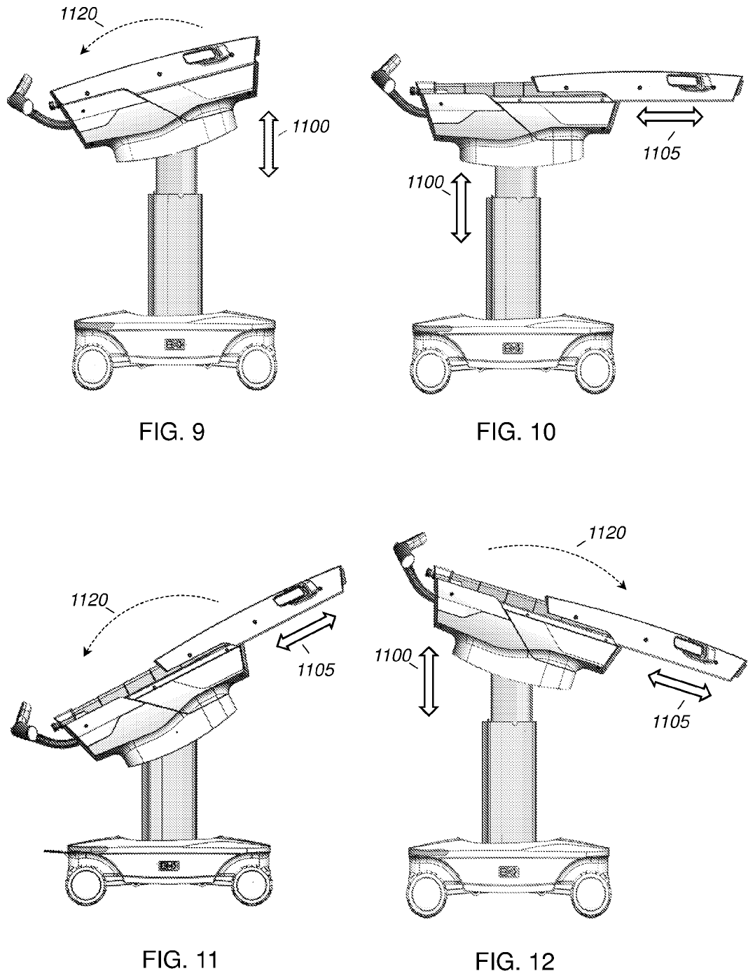

We now refer to FIGS. 9, 10, 11, 12, 13A and 13B.

In embodiments, it can be desirable to combine component movements (e.g., vertical and longitudinal displacement, pivoting) for alignment of the robotic-surgery device with a target vector. In the example of FIG. 9, a positioning system 500 is configured both for vertical displacement of the upper portion 510 as indicated by arrow 1100, and of pivoting of the upper portion 510 about the pitch-axis as indicated by arrow 1120. In some embodiments, the positioning system 500 is configured to allow and/or facilitate, e.g., by a common user input control, simultaneous vertical displacement and pivoting of the upper portion 510. In the example of FIG. 10, a positioning system 500 is configured to allow and/or enable both vertical displacement of the upper portion 510 as indicated by arrow 1100, and the longitudinal displacement of the slidable docking interface 515 as indicated by arrow 1105. In some embodiments, the positioning system 500 is configured to allow and/or facilitate, e.g., by a common user input control, simultaneous pivoting of the upper portion 510 and longitudinal displacement of the slidable docking interface 515. In the example of FIG. 11, a positioning system 500 is configured to allow and/or enable both pivoting of the upper portion 510 about the pitch-axis as indicated by arrow 1120 and the longitudinal displacement of the slidable docking interface 515 as indicated by arrow 1105. In some embodiments, the positioning system 500 is configured to allow and/or facilitate, e.g., by a common user input control, simultaneous pivoting of the upper portion 510 and longitudinal displacement of the slidable docking interface 515. In the example of FIG. 12, a positioning system 500 is configured to allow and/or enable vertical displacement of the upper portion 510 as indicated by arrow 1100, pivoting of the upper portion 510 about the pitch-axis as indicated by arrow 1120, and the longitudinal displacement of the slidable docking interface 515 as indicated by arrow 1105. In some embodiments, the positioning system 500 is configured to allow and/or facilitate, e.g., by a common user input control, simultaneous vertical displacement and pivoting of the upper portion 510 and longitudinal displacement of the slidable docking interface 515.

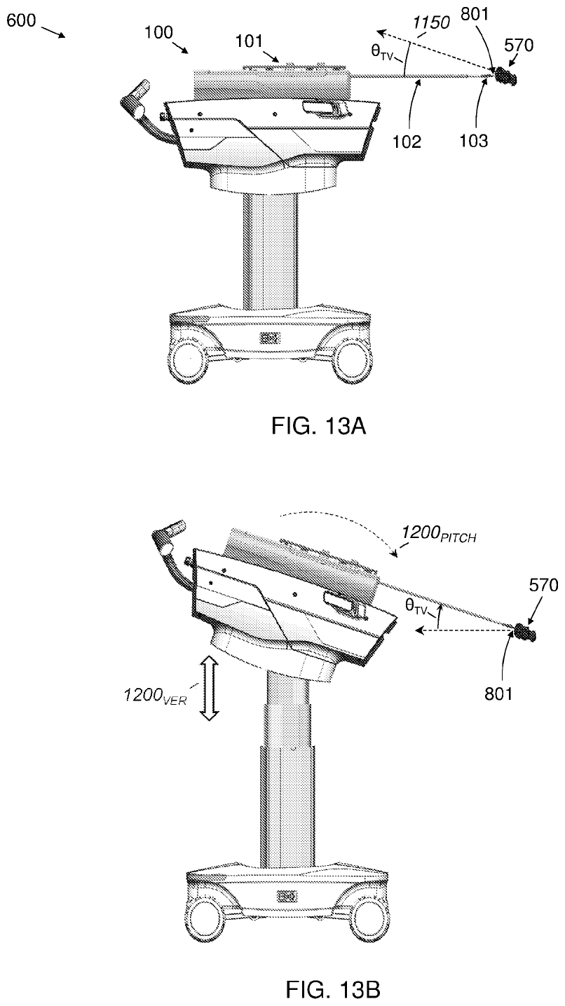

In embodiments, the aligning of the one or more surgical arms 102 of a robotic-surgery device 100 with a target vector, e.g., the target vector indicated by arrow 1150 in FIG. 13A, is performed with reference to a target location 801, and specifically by pivoting a robotic-surgery device 100 about the distal end 103 of the one or more surgical arms 102 of the robotic-surgery device 100, e.g., where the distal end 103 is disposed proximate to the target location 801. An illustrative example of a target location 801 is a proximal opening of a surgical access channel 570 placed to facilitate passage therethrough to a surgical site in a patient.

In a use-case example, respective distal ends 103 of the one or more surgical arms 102 are positioned at the target location 801, e.g., by suitable placement of the assembled-state positioning system 600. As shown in FIG. 13A, following said suitable placement, the one or more surgical arms 120 are seen to be oriented at an angle of rotation θ_{TV} from the target vector 1150. Aligning the one or more surgical arms 102 with the target vector 1150 is, according to embodiments, achievable by utilizing, in combination, the pivoting capability and vertical displacement capability of the upper portion 510 illustrated in FIG. 9. Referring again to FIG. 7, respective clockwise and counterclockwise arm-alignment controls 589_{FWD}, 589_{REV} are provided for controlling simultaneous pitching and vertical displacement of the upper portion 510 as respectively indicated in FIG. 13B by vertical displacement arrow 1200_{VER} and pitch arrow 1200_{PITCH}., in response to a user input received by the user-input panel 580. Optimally, the positioning system 500 includes electronic circuitry (not shown) configured, e.g., programmed, to simultaneously effect both the vertical displacement of the upper portion 510 and the pivoting of the upper portion 510 about the pitch axis. The electronic circuitry can be located within the user-input panel 580 or anywhere else within the positioning system 500. Alternatively, the electronic circuitry can include one or more processors located externally to the positioning system 500 and in electronic communication therewith. As illustrated in FIG. 13B, the electronic circuitry is effective to pivot the robotic-surgery device 100 about the distal end(s) 103 of the one or more surgical arms 102 through an angle θ_{TV} to align with the target vector 1150 while the distal end(s) remain at the target location 801. In embodiments, the electronic circuitry effects the pivoting by executing program instructions stored in or otherwise accessible to one or more processors of the electronic circuitry. In some embodiments, the pivot point is the distal end 103 of the one or more surgical arms 102, and in some embodiments, the pivot point is an end effector, e.g., a surgical, diagnostic or imaging tool, attached to the distal end 103. The control panel 580 and positioning system 500 can be configured such that the clockwise arm-alignment control 589_{FWD} is effective to cause an upwards vertical displacement of the upper portion 510 together with a clockwise pitching of the upper portion 510 (clockwise as seen in the perspective of FIG. 13B), in response to a user input received by the user-input panel 580, and such that the reverse arm-alignment control 589_{REV} is effective to cause an upwards vertical displacement of the upper portion 510 together with a ‘counterclockwise’ pitching (counterclockwise in the reference frame of the figures) of the upper portion 510, in response to a user input received by the user-input panel 580.

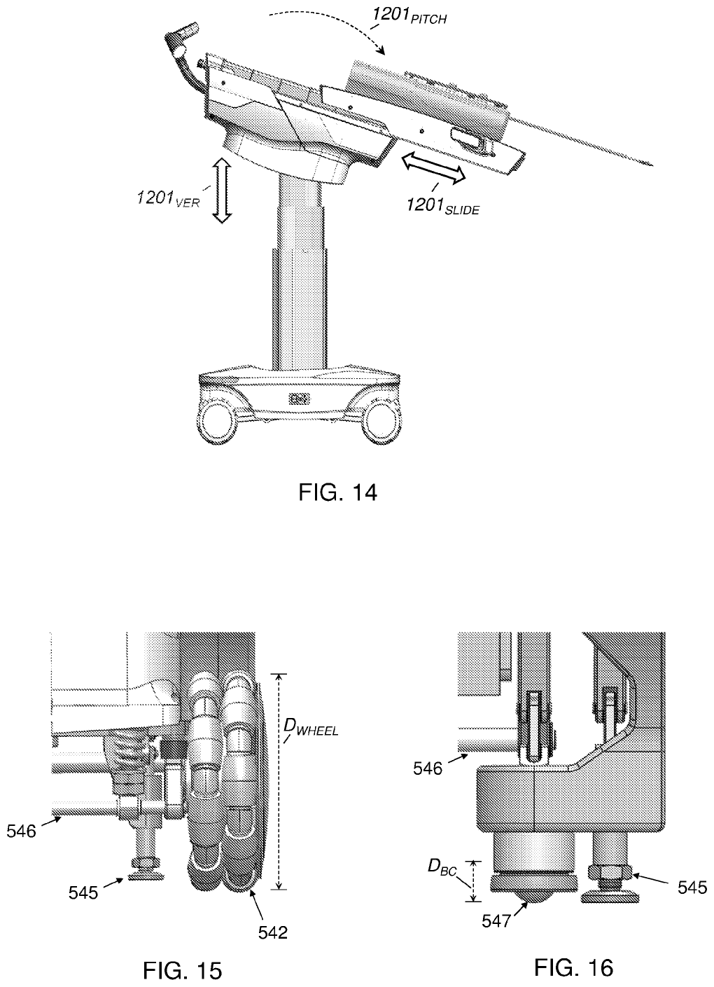

We now refer to FIG. 14. In a second use-case example, aligning the one or more surgical arms 102 with the target vector 1150 is achievable by utilizing, in combination, the pivoting capability and vertical displacement capability of the upper portion 510, together with the longitudinal displacement capability of the docking interface 515, illustrated in FIG. 12. In this example, respective clockwise and counterclockwise arm-alignment controls 589_{FWD}, 589_{REV} are configured for controlling simultaneous pitching and vertical displacement of the upper portion 510 as respectively indicated in FIG. 14 by vertical displacement arrow 1201_{VER} and pitch arrow 1201_{PITCH} together with simultaneous longitudinal displacement of the docking interface 515 as indicated in FIG. 14 by longitudinal displacement arrow 1201_{SLIDE}.

We now refer to FIGS. 15, 16 and 17.

In embodiments, a positioning system 500 (including, by extension, an assembled-state positioning system 600) is operable in respective positioning, position-adjusting, and stationary operating modes. Respective pluralities of different support elements are arranged to support the positioning system 500 in the different operating modes: respective pluralities of wheels 542, ball casters 547, and wheelless legs 545.

In a first operating mode available to the positioning system 500, the weight of the positioning system is supported by the plurality of wheels 542, which can include 3, 4, 5, 6 or more wheels, with the actual number chosen for convenience or to optimize distribution of weight, rolling resistance and/or stability. Wheels 542 can be independent or axled and are intended for primarily longitudinal (bi-directional) motion in positioning the positioning system 500, given that each wheel spins on a single axis, although longitudinal motion can include navigating arcs. In some embodiments, the wheels 542 are additionally configured to swivel, enabling some lateral movement as well. In an illustrative example shown in FIG. 15, a wheel 542 is designed to resist lateral movement. In some embodiments, the wheels 542 are powered, i.e., in electrical communication with one or motors of the positioning system for creating powered movement of the positioning system in the first operating mode.

In a second operating mode available to the positioning system 500, the weight of the positioning system 500 is supported by the plurality of ball casters 547, which can include 3, 4, 5, 6 or more ball casters, with the actual number chosen for convenience or to optimize distribution of weight, rolling resistance and/or stability. The number of ball casters 547 need not necessarily match the number of wheels 542. The ball casters are intended primarily for short-distance, omnidirectional movements of the positioning system 500. The ball casters 547 can be effective to enable precise movements necessary for fine-tuning, or adjusting, the position of the positioning system 500 achieved in the first operating mode. The precision is due, inter alia, to the ball casters 547 having smaller diameters than the wheels 542; in working examples, each of the wheels has a diameter, indicated by the arrow marked D_{WHEEL} in FIG. 15, that is at least double, or at least 3 times, or at least 4 times, or at least 5 times the diameter, indicated by the arrow marked D_{BC} in FIG. 16 of each of the ball casters 547. In examples, a diameter of a ball caster 547 is between 1 and 4 cm. In some examples, the ball-caster diameter is between 1 cm and 2 cm, between 1 cm and 2.5 cm, between 1 and 3 cm, between 1 and 3.5 cm, between 1.5 cm and 2.5 cm, between 1.5 cm and 3 cm, between 1.5 and 3.5 cm, between 1.5 and 4 cm, between 2 and 3 cm, between 2 and 3.5 cm, between 2 and 4 cm, between 2.5 and 3.5 cm, or between 2.5 and 4 cm. In examples, a diameter of a wheel is between 8 and 15 cm, between 8 and 12 cm, between 8 and 10 cm, between 10 and 15 cm, between 10 and 12 cm, or between 12 and 15 cm. In some example, a ratio of wheel diameters to ball caster diameters is between 3:1 and 8:1, between 3:1 and 7:1, between 3:1 and 6:1, between 3:1 and 5:1, between 4:1 and 8:1, between 4:1 and 7:1, between 4:1 and 6:1, between 5:1 and 8:1, or between 5:1 and 7:1.

Moreover, the ball casters 547 are more effective in producing precise lateral movements than even swiveled wheels due to the omnidirectional rotation of each ball caster about its own center.

In a third operating mode available to the positioning system 500, the weight of the positioning system 500 is supported by the plurality of wheelless legs 545, which can include 3, 4, 5, 6 or more legs, with the actual number chosen for convenience or to optimize distribution of weight and/or stability. The wheelless legs 545, in contrast to the wheels 542 and ball casters 547, are intended for preventing movement and rendering the positioning system 500 immobile. As such the legs 545 are designed to resist movement created by the various displacements and pivots of components discussed earlier in this disclosure.

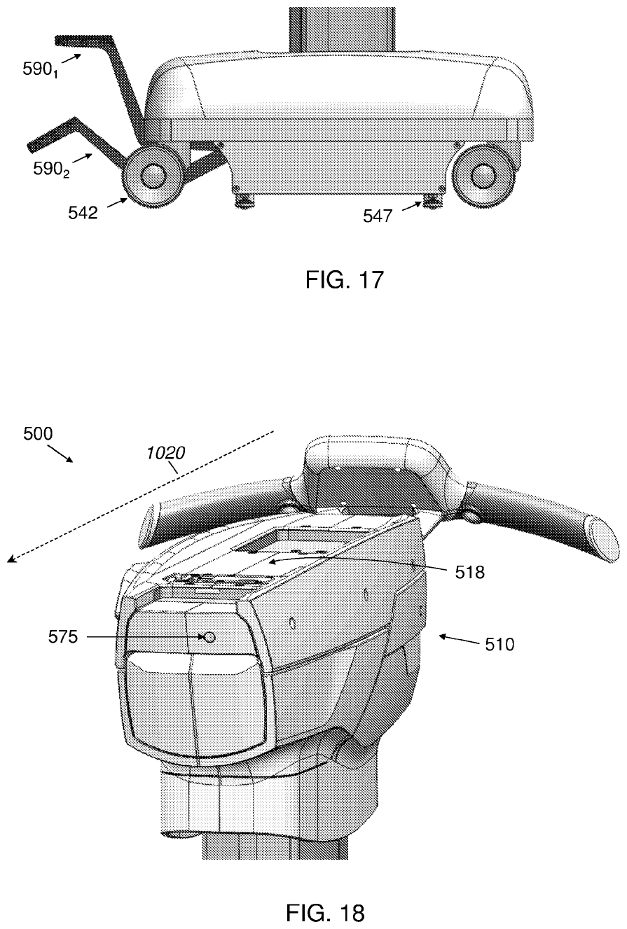

In embodiments, a plurality of mode-change pedals 590 are provided on the positioning system 500, as can be seen in FIGS. 6A, 6B and 8. In a first mode-change example, an application of a pedal 590 with the positioning system 500 supported by the plurality of wheels 542 is effective to raise the wheels 542 and transfer support of the positioning system 500 to the plurality of ball casters 547, as illustrated schematically in FIG. 17. In a second mode-change example, an application of a pedal 590 with the positioning system 500 supported by the plurality of wheels 542 is effective to lower the ball casters 547 and transfer support of the positioning system 500 to the plurality of ball casters 547. In a third mode-change example, an application of a pedal 590 with the positioning system 500 supported by the plurality of wheels 542 is effective to raise the wheels 542 and transfer support of the positioning system 500 to the plurality of wheelless legs 545. In a fourth mode-change example, an application of a pedal 590 with the positioning system 500 supported by the plurality of wheels 542 is effective to lower the wheelless legs 545 and transfer support of the positioning system 500 to the plurality of wheelless legs 545. In a fifth mode-change example, an application of a pedal 590 with the positioning system 500 supported by the plurality of ball casters 547 is effective to transfer support of the positioning system 500 to the plurality of wheelless legs 545. In a sixth node-change example, an application of a pedal 590 with the positioning system 500 supported by the plurality of wheels 542 is effective to lower the ball casters 547 and transfer support of the positioning system 500 to the plurality of ball casters 547. In a seventh node-change example, an application of a pedal 590 with the positioning system 500 supported by the plurality of wheels 542 is effective to lower the wheelless legs 545 and transfer support of the positioning system 500 to the plurality of wheelless legs 545.

In embodiments, the one or more mode-change pedals 590 comprise a first mode-change pedal 590₁ and a second mode-change pedal 590₂, arranged so that when the positioning system 500 is supported by the plurality of wheels 542, an application of the first mode-change pedal 590₁ is effective to transfer support of the positioning system 500 from the plurality of wheels 542 to the plurality of ball casters 547, and an application of the second mode-change pedal 590₂ is effective to transfer support of the positioning system 500 from the plurality of ball casters 547 to the plurality of wheelless legs 545.

In a non-limiting example, an assembled-state positioning system 600 is moved longitudinally in a first operating mode for a distance of over 50 meters. Upon an application of a mode-change pedal 590, support of the assembled-state positioning system 600 is transferred to the plurality of ball casters 547. The position of the assembled-state positioning system 600 is adjusted over distances of less than 2 meters in both longitudinal and lateral directions so as to bring the distal end 103 of a surgical arm 102 proximally seated in the motor-control unit 101 of the robotic-surgery device to a target location.

We now refer to FIGS. 18-21.

In embodiments, alignment of the surgical arms 102 with a target vector, e.g., the target vector 1150 of FIG. 13A, is accomplished by using a visual aid. The visual aid can include alignment tools such as optical sights or iron sights. Use of the optical sights or iron sights can be enhanced by using an illumination system to illuminate a target such as a visual alignment of sight elements.

FIG. 18 is a proximal-perspective detail view of a positioning system 500 showing the upper portion 510. An illumination source 575 is provided to illuminate in a positioning-system-distal direction, the direction indicated by arrow 1020 in FIG. 18.

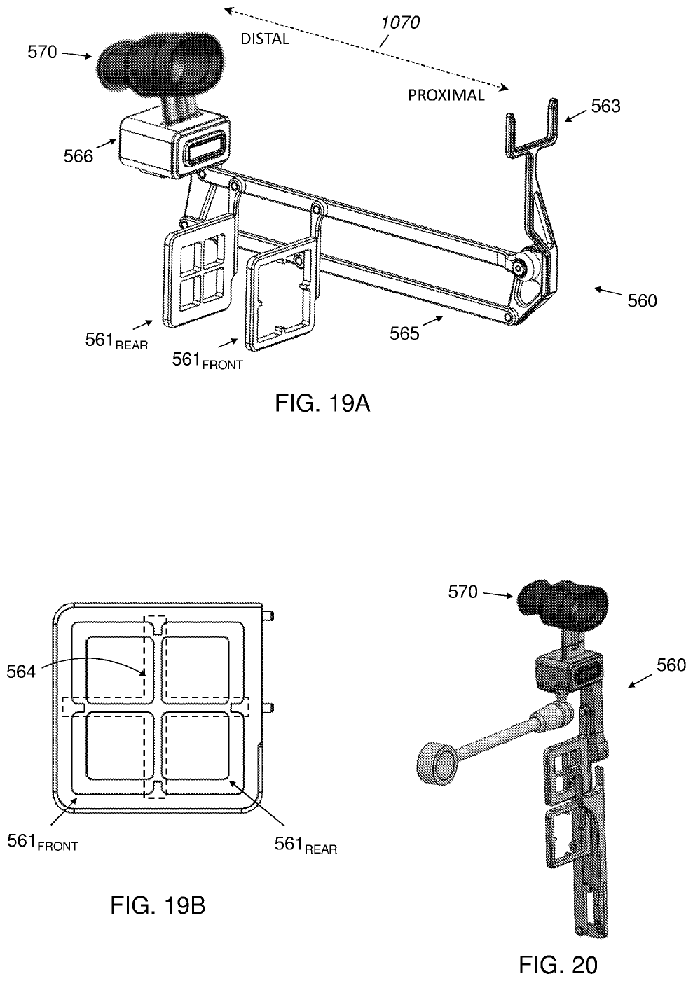

FIG. 19A shows a perspective view of an access-channel-assembly frame 560 to be used, together with the illumination source 575 of the positioning system 500, as a visual aid. The access-channel-assembly frame 560 includes a frame-member assembly 565 for stabilizing the structure of the frame 560. A mounting block 566 for mounting, e.g., coupling to, a surgical access channel 570 is attached to the frame-member assembly 565 at a distal end of the frame 560, ‘distal’ being the direction indicated by arrow 1070 in FIG. 19A. A pair of iron sights 561_{REAR}, 561_{FRONT} is attached to the frame-member assembly 565. ‘Iron sights’ is a term of art meaning non-optical sights, without prejudice to construction materials. Iron sights 561, along with the frame-member assembly 565, are fabricated from any suitable material such as a copper alloy or a stainless steel. In some embodiments, an alignment arm 563 is provided for assistance in visually aligning the one or more surgical arms 102 with the surgical access channel 570. The iron sights 561 can be either fixed or ‘fold-out’, i.e., configured to fold out for use and to be folded back to the frame-member assembly 565 when not in use, e.g., after a precise alignment with the target vector 1150 or the surgical access channel 570 has been achieved. FIG. 20 schematically illustrates an example of a folded-up access-channel-assembly frame 560 with the iron sights 561 folded back.

The first and second iron sights 561_{REAR}, 561_{FRONT} are aligned with each other in an iron-sight-distal direction parallel to arrow 1070, so as to define an illumination target 564, which is shown in FIG. 19B as a visual convergence of the center portions of the front and rear iron sights 561. The specific shape of the iron sights 561 and of their visual convergence or alignment, i.e., the illumination target, is entirely a matter of design choice, and any suitable sight shape or target shape can be implemented. In an example, the illumination source 575, which can be, for example a laser illuminator or a focused or masked source of incoherent light, is configured to project a shaped beam that illuminates both iron sights 561_{REAR}, 561_{FRONT} within the outline of the illumination target 564. As shown in FIG. 7, an illumination-source control 562 can be placed on the user-input panel 580 for activating, regulating and/or deactivating the illumination target.

In embodiments, the illumination target 564, e.g., the center of the illumination target 564, is offset from the surgical access channel 570 when the access-channel-assembly frame 560 is mounted to the mounting block 566. The offset includes an offset in the iron-sight-distal direction (parallel to arrow 1070) and an offset in a transverse direction orthogonal to the iron-sight-distal direction.

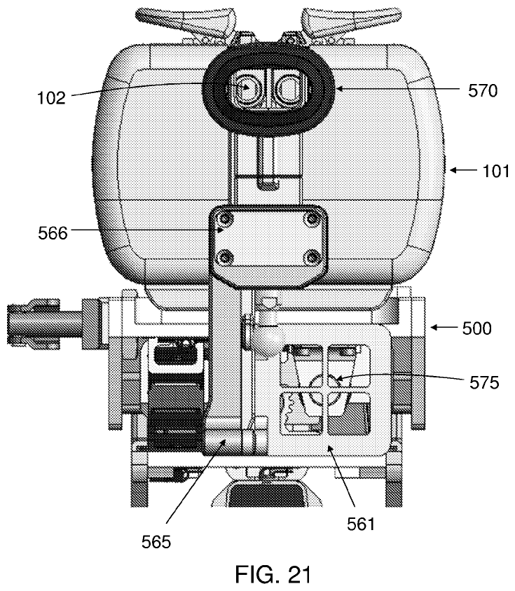

FIG. 21 includes a schematic, partial distal elevation view of an assembled-state positioning system 600 aligned with an access-channel-assembly frame 560; specifically, the frame-member assembly 565, iron sights 561, and mounting block 566 with surgical access channel 570 mounted thereto are visible in the ‘foreground’, i.e., the distal perspective is from a point distal of the access-channel-assembly frame 560 which in turn is distal of the assembled-state positioning system 600.

The skilled artisan will understand that for this perspective to be achieved, (i) the one or more surgical arms are aligned with the target vector 1150 (via the surgical access channel 570); (ii) the iron-sight-distal direction (arrow 1070) of FIG. 19A and the orthogonal positioning-system-distal direction (arrow 1020 of FIG. 18) are parallel; and (iii) the angle of view of FIG. 21 is aligned with the target vector 1150.

This perspective is effective to show that: (i) the illumination target 564, i.e., the center of the illumination target 564, is offset from the surgical access channel 570 at least in a transverse direction orthogonal to the iron-sight-distal direction; (ii) the illumination source 575 is offset from the one or more surgical arms 102, at least in a transverse direction orthogonal to the positioning-system-distal direction, which, in FIG. 21, is parallel to the iron-sight-distal direction as discussed above; and (iii) the transverse offset of the illumination target 564 from the surgical access channel 570 is equal, in magnitude and direction, to the transverse offset of the illumination source 575 from the one or more surgical arms 102. The equality of the offsets means that in the configuration of FIG. 21, correctly illuminating the illumination target 564 indicates that the one or more surgical arms 102 are, in fact, aligned with the target vector 1150. In embodiments, the two offsets are ‘substantially’ equal, which means that the offsets are within ±1% of each other, or within ±2% of each other, or within ±3% of each other, or within ±4% of each other, or within ±5% of each other, or within ±6% of each other, or within ±7% of each other, or within ±8% of each other, or within ±9% of each other, or within ±10% of each other, or within ±11% of each other, or within ±12% of each other, or within ±13% of each other, or within ±14% of each other, or within ±15% of each other.

While the preceding embodiments have been discussed with respect to the use of iron sights for employing a visual aid, the alternative or additional use of one or more optical sights, with or without an illumination source, is also within the scope of the invention.

In embodiments, any one of the positioning systems 500 illustrated in and discussed with respect to FIGS. 1A, 1B, 6A, 6B, 9, 10, 11, 12, and 18 can include and/or be provided with any one or more, or all, of the following common features and capabilities, and not exhaustively: vertical displacing of the upper portion 510; pivoting about a pitch-axis member of the upper portion 510; longitudinal displacement of the docking interface 515; any combination of two or more of the foregoing displacing and pivoting capabilities; operation of the positioning system in any one of the respective positioning, position-adjusting, and stationary operating modes; and transferring of support of the positioning system 500 among the respective pluralities of different support elements, i.e., respective pluralities of wheels 542, ball casters 547, and wheelless legs 545 using one or more mode-change pedals 590.

In embodiments, any one of the assembled-state positioning systems 600 illustrated in and discussed with respect to FIGS. 2, 3, 4A, 4B, 5, 13A, 13B, 14, and 21 can include and/or be provided with any one or more, or all, of the following common features and capabilities, and not exhaustively: vertical displacing of the upper portion 510; pivoting about a pitch-axis member of the upper portion 510; longitudinal displacement of the docking interface 515; any combination of two or more of the foregoing displacing and pivoting capabilities; operation of the positioning system in any one of the respective positioning, position-adjusting, and stationary operating modes; transferring of support of the positioning system 500 among the respective pluralities of different support elements, i.e., respective pluralities of wheels 542, ball casters 547, and wheelless legs 545 using one or more mode-change pedals 590; pivoting of the robotic-surgery device 100 about the distal end 103 (and or an end effector coupled to the distal end 103) of one or more surgical arms 102 proximally seated in the motor-control unit 101 of the robotic-surgery device 100; and use of a visual aid, e.g., an illumination source 575 and sights 561, for aligning the one or more surgical arms with a target vector 1150, including a target vector that describes at least one of a position of a surgical access channel 570 and an orientation of a surgical access channel 570.

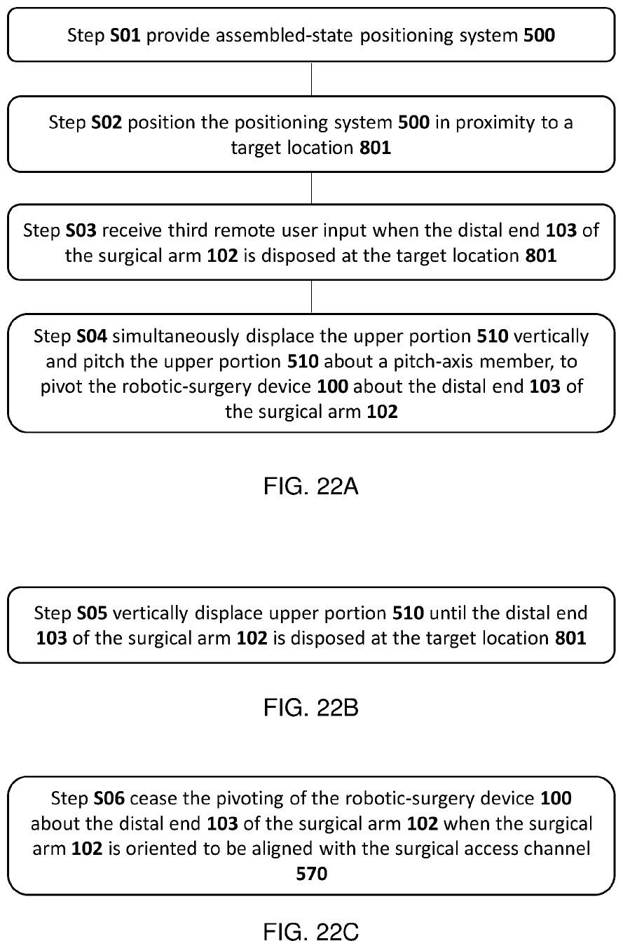

A method is disclosed, according to embodiments. As shown in the flowchart of FIG. 22A, the method comprises any or all of the following steps:

Step S01: providing an assembled-state positioning system 600, i.e., the positioning system 500 according to any of the embodiments disclosed hereinabove in an assembled state in which the motor-control unit 101 is secured to the docking interface 515 and one or more surgical arms 102 are proximally seated in respective surgical-arm-receiving volumes of the motor-control unit 101.

Step S02: positioning the positioning system 500 in proximity to a target location 801, i.e., with the distal tip 103 of the one or more surgical arms 102 in proximity to a target location 801. In some embodiments, the target location 801 is proximate to a surgical access channel 570.

Step S03: receiving a remote user input respective of simultaneously displacing the upper portion 510 vertically and pitching the upper portion 510 about a pitch-axis member, for pivoting the robotic-surgery device 100 about the distal end 103 of the surgical arm 102 when the distal end 103 of the surgical arm 102 is disposed at the target location 801.

Step S04: simultaneously displacing the upper portion 510 vertically and pitching the upper portion 510 about a pitch-axis member, so as to pivot the robotic-surgery device 100 about the distal end 103 of the surgical arm 102.

In some embodiments, as shown in the flowchart of FIG. 22B, the method additionally comprises, after Step S02:

Step S05 vertically displacing the upper portion 510 until the distal end 103 of the surgical arm 102 is disposed at the target location 801. This step can be useful if the positioning of Step S02 brings the distal tip 103 in proximity to the target location 801 but with a vertical offset.

In some embodiments, as shown in the flowchart of FIG. 22C, the method additionally comprises, after Step S04:

Step S06 ceasing the pivoting of the robotic-surgery device 100 about the distal end 103 of the surgical arm 102 when the surgical arm 102 is oriented to be aligned with the surgical access channel 570.

In some embodiments, the ceasing of Step S06 is carried out in response to an indication of a visual aid that the one or more surgical arms 102 are oriented to be aligned with the target vector 1150 and/or with the surgical access channel 570.

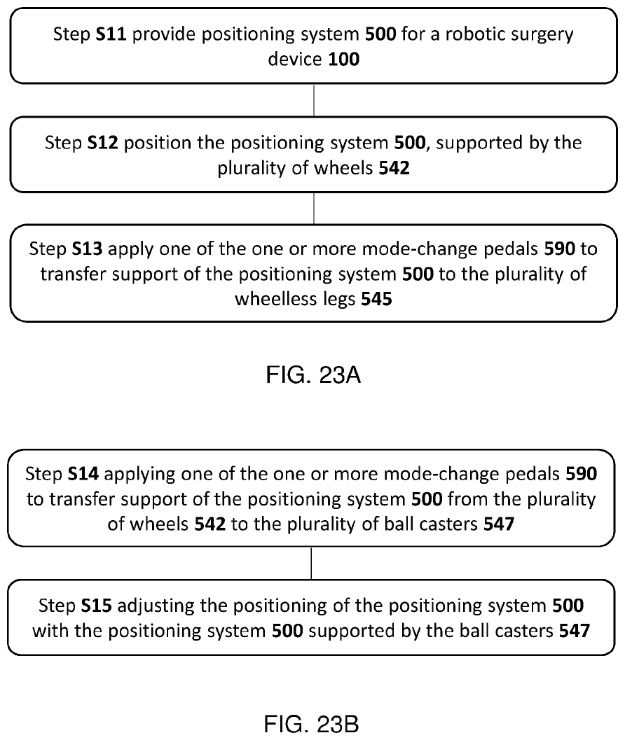

A method is disclosed, according to embodiments. As shown in the flowchart of FIG. 23A, the method comprises any or all of the following steps:

Step S11 providing positioning system 500 for a robotic surgery device 100. The positioning system 500 comprises: (i) a plurality of wheels 542 arranged to support the positioning system 500 in a positioning operating mode; (ii) a plurality of ball casters 547 arranged to support the positioning system 500 in a position-adjusting operating mode; (iii) a plurality of wheelless legs 545 arranged to support the positioning system 500 in a stationary operating mode; and (iv) one or more mode-change pedals 590 configured to transfer support of the positioning system 500 from one respective plurality of support elements 542, 545, 547 to another. In some embodiments, the positioning system 500 is provided a part of an assembled-state positioning system 600.

Step S12 positioning the positioning system 500, supported by the plurality of wheels 542.

Step S13 applying one of the one or more mode-change pedals 590 to transfer support of the positioning system 500 to the plurality of wheelless legs 545, thereby placing the positioning system 500 in the stationary operating mode.

In some embodiments, as shown in the flowchart of FIG. 23B, the method additionally comprises, after Step S12:

Step S14 applying one of the one or more mode-change pedals 590 to transfer support of the positioning system 500 from the plurality of wheels 542 to the plurality of ball casters 547.

Step S15 adjusting the positioning of the positioning system 500, with the positioning system 500 supported by the ball casters 547.

In some embodiments of the method, at least one of the one or more mode-change pedals is arranged to lift the plurality of wheels when applied.

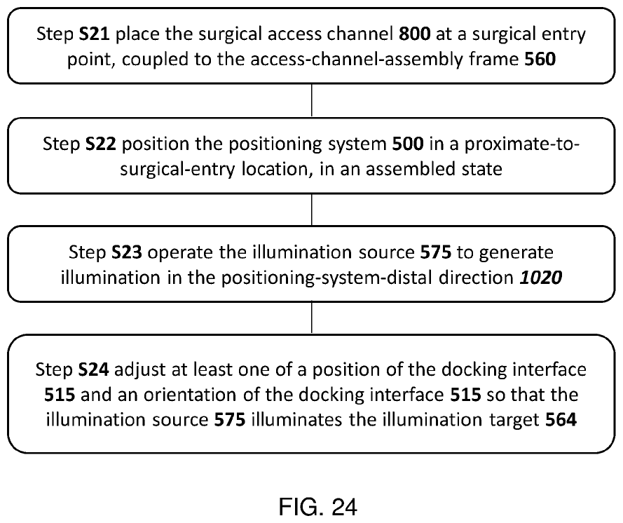

A method for employing a medical apparatus is disclosed, according to embodiments. The medical apparatus includes a positioning system 500 according to any of the embodiments disclosed herein, an illumination source 575, and an access-channel-assembly frame 560 according to any of the embodiments disclosed herein. According to the method, the illumination source 575 is coupled to the docking interface 515—either directly or indirectly, e.g., via the upper portion 510—and operable to generate illumination in a positioning-system-distal direction. Further, the illumination source 575 is offset from the one or more surgical arms 102, or from respective surgical-arm-receiving volumes, at least in a transverse direction that is orthogonal to the positioning-system-distal direction. According to the method, the access-channel-assembly frame 560 comprises first and second iron sights 561 aligned with each other to define an illumination target and an iron-sight-distal direction. Further, the access-channel-assembly frame 560 is configured to have coupled thereto a surgical access channel 570 shaped to allow traversal of respective distal portions 103 of one or more surgical arms 102 of a robotic-surgery device 100. The coupling of the surgical access channel 570 is such that the first and second iron sights 561 are offset from the coupled surgical access channel 570 at least in a transverse direction that is orthogonal to the iron-sight-distal direction. As shown in the flowchart of FIG. 24, the method comprises any or all of the following steps:

Step S21 placing the surgical access channel 800 at a surgical entry point, coupled to the access-channel-assembly frame 560.

Step S22 positioning the positioning system 500 in a proximate-to-surgical-entry location, in an assembled state in which the motor-control unit 101 is mounted to the docking interface 515 of the positioning system 500, and the one or more surgical arms 102 are proximally received in respective arm-receiving volumes of the motor-control unit 101, i.e., the positioning system 500 is positioned as part of an assembled-state positioning system 600.

Step S23 operating the illumination source 575 to generate illumination in the positioning-system-distal direction 1020.

Step S24 adjusting at least one of a position of the docking interface 515 and an orientation of the docking interface 515 so that the illumination source 575 illuminates the illumination target 564. In some embodiments, the adjusting of Step S24 is effective to align the one or more surgical arms 102 for passage of distal ends 103 thereof through the surgical access channel 570.

Any of the method steps disclosed herein can be combined in any useful way with other method steps within the scope of the present invention.

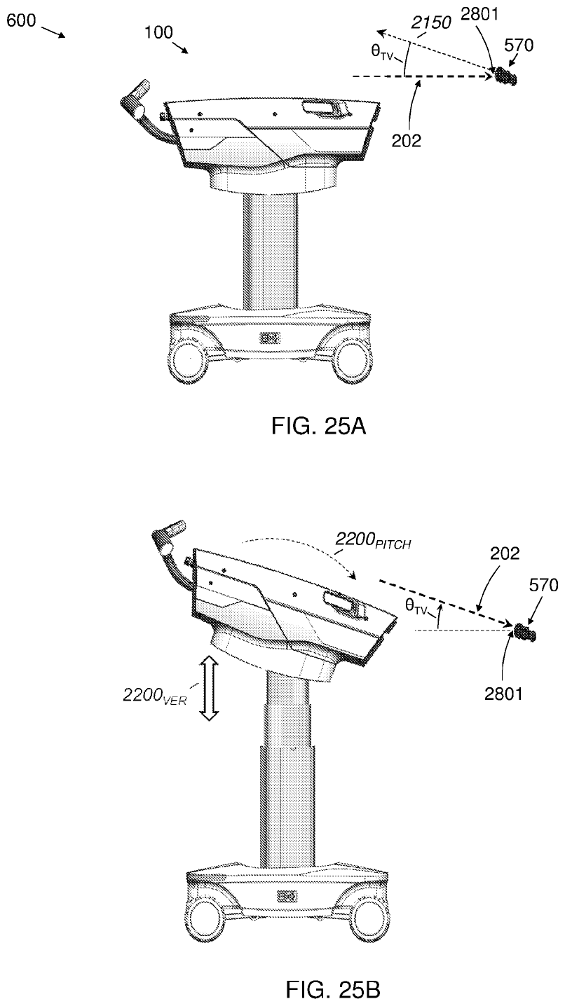

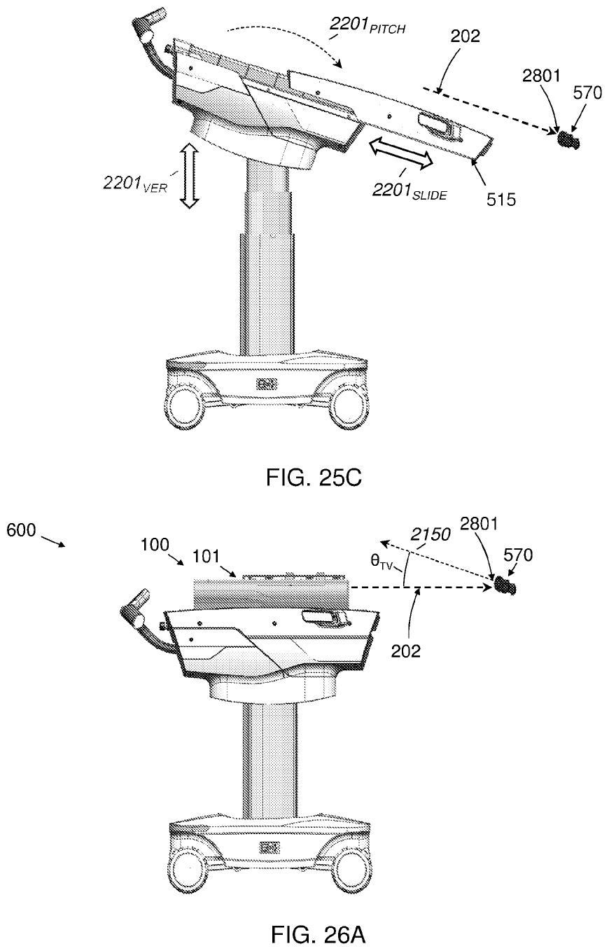

We now refer to FIGS. 25A, 25B and 25C.

In embodiments, aligning of an operative vector of a robotic-surgery device 100, or more specifically, an operative vector 202 of the motor-control unit 101, with a target vector, e.g., the target vector indicated by arrow 2150 in FIG. 25A, can be performed using one or more the controllable movements disclosed herein: vertical displacement of an upper portion 510 of the positioning system 500, pitch-axis pivoting of the upper portion 510, and longitudinal displacement of a the slidable docking interface 515. In an example, such an aligning can be performed during set up of a robotic-surgery device 100 for a surgical operation without the robotic-surgery device 100 installed on the slidable docking interface 515 of the positioning system 500 as shown in FIG. 25A.

The aligning of the operative vector with the target vector can be performed with reference to a remote target point 2801, and specifically by pivoting the upper portion 510 of the positioning system 500 about the remote target point. An illustrative example of a target location 2801 is a proximal opening of a surgical access channel 570 placed to facilitate passage therethrough to a surgical site in a patient.

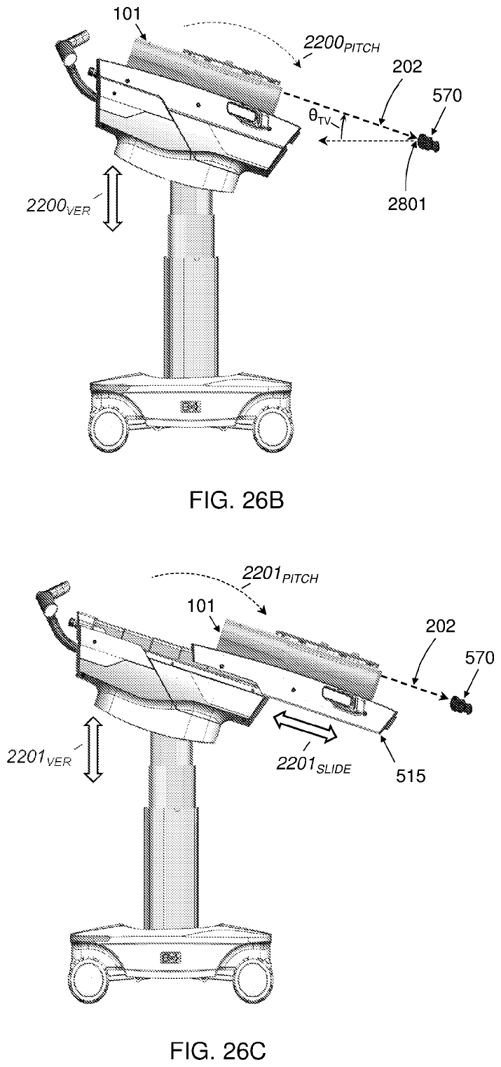

As shown in FIG. 25A, following suitable placement of the positioning system 500, an operative vector 202 of the motor-control unit 101 (whether present as in FIGS. 26A-C, or not present as in FIGS. 25A-C) is seen to be oriented at an angle of rotation θ_{TV} from the target vector 2150. Aligning the operative vector 202 with the target vector 2150 is, according to embodiments, achievable by utilizing, in combination, the pivoting capability and vertical displacement capability of the upper portion 510 illustrated, e.g., in FIG. 9. In some embodiments, the operative vector is a vector that aligns with an expected vector of a surgical arm 102 when installed in the motor-control unit 101. Referring again to FIG. 7, respective forward (‘clockwise’) and reverse (‘counterclockwise’) arm-alignment controls 589_{FWD}, 589_{REV} are provided for controlling simultaneous pitching and vertical displacement of the upper portion 510 as respectively indicated in FIG. 25B by vertical displacement arrow 2200_{VER} and pitch arrow 2200_{PITCH}, e.g., in response to a user input received by the user-input panel 580. Optimally, the positioning system 500 includes electronic circuitry (not shown) configured, e.g., programmed, to simultaneously effect both the vertical displacement of the upper portion 510 and the pivoting of the upper portion 510 about a pitch axis. The electronic circuitry can be located within the user-input panel 580 or anywhere else within the positioning system 500. Alternatively, the electronic circuitry can include one or more processors located externally to the positioning system 500 and in electronic communication therewith. As illustrated in FIG. 25B, the electronic circuitry is effective to pivot the operative vector 202 about the remote target point 2801 through an angle θ_{TV} to align with the target vector 2150. In embodiments, the electronic circuitry effects the pivoting by executing program instructions stored in or otherwise accessible to one or more processors of the electronic circuitry. The control panel 580 and positioning system 500 can be configured such that the forward (clockwise) arm-alignment control 589_{FWD} is effective to cause an upwards vertical displacement of the upper portion 510 together with a clockwise pitching of the upper portion 510 (clockwise as seen in the perspective of FIG. 25B), in response to a user input received by the user-input panel 580, and such that the reverse arm-alignment control 589_{REV} is effective to cause an upwards vertical displacement of the upper portion 510 together with a ‘counterclockwise’ pitching (counterclockwise in the reference frame of the figures) of the upper portion 510, in response to a user input received by the user-input panel 580.

In some embodiments, the aligning of the operative vector 202 with the target vector 2150 is accomplished in a single step. In some embodiments, the aligning of the operative vector 202 with the target vector 2150 is accomplished in two steps: (i) lining up the operative vector 202 to intercept the remote target point 2801 and (ii) pivoting the docking interface—with or without the motor-control unit 101—about the remote target point 2801 to align the operative vector 202 of the motor-control unit 101 with a target vector 2150 at the remote target point 2801.

We now refer to FIG. 25C. In a second use-case example, aligning the operative vector 202 with the target vector 2150 is achievable by utilizing, in combination, the pivoting capability and vertical displacement capability of the upper portion 510, together with the longitudinal displacement capability of the docking interface 515, e.g., as illustrated in FIG. 12. In this example, respective clockwise and counterclockwise arm-alignment controls 589_{FWD}, 589_{REV} are configured for controlling simultaneous pitching and vertical displacement of the upper portion 510 as respectively indicated in FIG. 25C by vertical displacement arrow 2201_{VER} and pitch arrow 2201_{PITCH} together with simultaneous longitudinal displacement of the docking interface 515 as indicated in FIG. 25C by longitudinal displacement arrow 2201_{SLIDE}.

In embodiments, the pivoting capabilities described with reference to FIGS. 25A-C can be combined with the visual alignment system of FIGS. 19A-B such that aligning the operative vector with the target vector can include aligning an offset vector with an offset target vector.

We now refer to FIGS. 26A, 26B and 26C.

In embodiments, aligning of an operative vector of a robotic-surgery device 100, or more specifically, an operative vector 202 of the motor-control unit 101, with a target vector, e.g., the target vector indicated by arrow 2150 in FIG. 26A, can be performed using one or more the controllable movements disclosed herein: vertical displacement of an upper portion 510 of the positioning system 500, pitch-axis pivoting of the upper portion 510, and longitudinal displacement of a the slidable docking interface 515. In an example, such an aligning can be performed during set up of a robotic-surgery device 100 for a surgical operation, with the motor-control unit 101 of the robotic-surgery device 100 installed on the slidable docking interface 515 of the positioning system 500 as shown in FIG. 26A. The aligning of the operative vector with the target vector can be performed with reference to a remote target point 2801, and specifically by pivoting the upper portion 510 of the positioning system 500 about the remote target point. An illustrative example of a target location 2801 is a proximal opening of a surgical access channel 570 placed to facilitate passage therethrough to a surgical site in a patient.

As shown in FIG. 26A, following suitable placement of the positioning system 500, an operative vector 202 is seen to be oriented at an angle of rotation θ_{TV} from the target vector 2150. Aligning the operative vector 202 with the target vector 2150 is, according to embodiments, achievable by utilizing, in combination, the pivoting capability and vertical displacement capability of the upper portion 510 illustrated, e.g., in FIG. 9. In some embodiments, the operative vector is a vector that aligns with an expected vector of a surgical arm 102 when installed in the motor-control unit 101. Referring again to FIG. 7, respective forward (‘clockwise’) and reverse (‘counterclockwise’) arm-alignment controls 589_{FWD}, 589_{REV} are provided for controlling simultaneous pitching and vertical displacement of the upper portion 510 as respectively indicated in FIG. 26B by vertical displacement arrow 2200_{VER} and pitch arrow 2200_{PITCH}, e.g., in response to a user input received by the user-input panel 580. Optimally, the positioning system 500 includes electronic circuitry (not shown) configured, e.g., programmed, to simultaneously effect both the vertical displacement of the upper portion 510 and the pivoting of the upper portion 510 about a pitch axis. The electronic circuitry can be located within the user-input panel 580 or anywhere else within the positioning system 500. Alternatively, the electronic circuitry can include one or more processors located externally to the positioning system 500 and in electronic communication therewith. As illustrated in FIG. 25B, the electronic circuitry is effective to pivot the operative vector 202 about the remote target point 2801 through an angle θ_{TV} to align with the target vector 2150. In embodiments, the electronic circuitry effects the pivoting by executing program instructions stored in or otherwise accessible to one or more processors of the electronic circuitry. The control panel 580 and positioning system 500 can be configured such that the forward (clockwise) arm-alignment control 589_{FWD} is effective to cause an upwards vertical displacement of the upper portion 510 together with a clockwise pitching of the upper portion 510 (clockwise as seen in the perspective of FIG. 26B), in response to a user input received by the user-input panel 580, and such that the reverse arm-alignment control 589_{REV} is effective to cause an upwards vertical displacement of the upper portion 510 together with a ‘counterclockwise’ pitching (counterclockwise in the reference frame of the figures) of the upper portion 510, in response to a user input received by the user-input panel 580.

In some embodiments, the aligning of the operative vector 202 with the target vector 2150 is accomplished in a single step. In some embodiments, the aligning of the operative vector 202 with the target vector 2150 is accomplished in two steps: (i) lining up the operative vector 202 to intercept the remote target point 2801 and (ii) pivoting the docking interface—with or without the motor-control unit 101—about the remote target point 2801 to align the operative vector 202 of the motor-control unit 101 with a target vector 2150 at the remote target point 2801.

We now refer to FIG. 26C. In a second use-case example, aligning the operative vector 202 with the target vector 2150 with the motor-control unit 101 installed on the docking interface 515 is achievable by utilizing, in combination, the pivoting capability and vertical displacement capability of the upper portion 510, together with the longitudinal displacement capability of the docking interface 515, e.g., as illustrated in FIG. 12. In this example, respective clockwise and counterclockwise arm-alignment controls 589_{FWD}, 589_{REV} are configured for controlling simultaneous pitching and vertical displacement of the upper portion 510 as respectively indicated in FIG. 26C by vertical displacement arrow 2201_{VER} and pitch arrow 2201_{PITCH} together with simultaneous longitudinal displacement of the docking interface 515 as indicated in FIG. 25C by longitudinal displacement arrow 2201_{SLIDE}.

For the present disclosure, the term ‘electronic circuitry’ is intended broadly to describe any combination of hardware, software and/or firmware. Electronic circuitry may include any executable code module (i.e., stored on a computer-readable medium) and/or firmware and/or hardware element(s) including but not limited to field programmable logic array (FPLA) element(s), hard-wired logic element(s), field programmable gate array (FPGA) element(s), and application-specific integrated circuit (ASIC) element(s). Any instruction set architecture may be used including but not limited to reduced instruction set computer (RISC) architecture and/or complex instruction set computer (CISC) architecture. Electronic circuitry may be located in a single location or distributed among a plurality of locations where various circuitry elements may be in wired or wireless electronic communication with each other.

It will be appreciated by persons skilled in the art that the present invention is not limited to what has been particularly shown and described hereinabove. Rather, the scope of the present invention includes both combinations and sub-combinations of the various features described hereinabove, as well as variations and modifications thereof that are not in the prior art, which would occur to persons skilled in the art upon reading the foregoing description.

1. A positioning system for a robotic-surgery device, the positioning system comprising:
a. a lower portion comprising a pillar section extending from a wheeled base;
b. an upper portion supported by the pillar section and comprising a docking interface adapted for securing thereto a motor-control unit of a robotic-surgery device, the upper portion configured for displacing vertically in response to a first remote user input and for pitching, in response to a second remote user input, about a pitch-axis member mediating between the pillar section and the upper-portion; and
c. electronic circuitry programmed to cause the upper portion to simultaneously displace vertically and pitch about the pitch-axis member, in response to a third remote user input, so as to pivot a robotic-surgery device secured to the docking interface about a distal end of a surgical arm proximally seated in a surgical-arm-receiving volume of the motor-control unit.

2. The positioning system of claim 1, wherein the electronic circuitry is programmed to effect the pivoting of the robotic-surgery device about the distal end of the surgical arm without displacing the distal end of the surgical arm.

3. The positioning system of either one of claim 1 or 2, wherein the distal end of the surgical arm is characterized by having an end-effector coupled thereto, and the pivoting of the robotic-surgery device about the distal end of the surgical arm includes pivoting the robotic-surgery device about a distal end of the end-effector.

4. The positioning system of any one of the preceding claims, additionally comprising a user-input device arranged to receive the first, second and third remote user inputs from a user.

5. The positioning system of any one of the preceding claims, wherein the docking interface is configured for displacing longitudinally in response to a fourth remote user input.

6. The positioning system of claim 5, wherein the user-input device is additionally arranged to receive the fourth remote user input from the user.

7. The positioning system of either one of claim 5 or 6, wherein the electronic circuitry is additionally programmed to cause the docking interface to displace longitudinally simultaneously with the vertical displacing and pitching of the upper portion, in response to a fifth remote user input.

8. The positioning system of any one of the preceding claims, additionally comprising a visual aid for aligning the surgical arm with a target vector.

9. The positioning system of claim 8, wherein the target vector describes at least one of a position of a surgical access channel and an orientation of a surgical access channel

10. The positioning system of any one of the preceding claims, wherein the pillar section is at least partly telescopic.

11. The positioning system of any one of the preceding claims, wherein the positioning system is configured to provide electrical power to the motor-control unit.

12. A method comprising:
a. providing the positioning system of any one of the preceding claims in an assembled state in which the motor-control unit is secured to the docking interface and a surgical arm is proximally seated in a surgical-arm-receiving volume of the motor-control unit;
b. positioning the positioning system in proximity to a target location;
c. receiving the third remote user input when the distal end of the surgical arm is disposed at the target location; and
d. in response to receiving the third remote user input, simultaneously displacing the upper portion vertically and pitch the upper portion about the pitch-axis member, so as to pivot the robotic-surgery device about the distal end of the surgical arm.

13. The method of claim 12, additionally comprising, after the positioning: vertically displacing the upper portion until the distal end of the surgical arm is disposed at the target location

14. The method of either one of claim 12 or 13, wherein the target location is proximate to a surgical access channel.

15. The method of claim 14, additionally comprising: ceasing the pivoting of the robotic-surgery device about the distal end of the surgical arm when the surgical arm is oriented to be aligned with the surgical access channel.

16. The method of claim 15, wherein the ceasing is in response to an indication of a visual aid that the surgical arm is oriented to be aligned with the surgical access channel.

17. The method of claim 16, wherein the visual aid includes a laser illuminator.

18. A positioning system for a robotic-surgery device, the positioning system comprising:
a. a lower portion comprising a pillar section extending from a wheeled base;
b. an upper portion supported by the pillar section and comprising a docking interface adapted for securing thereupon a motor-control unit of a robotic-surgery device, the upper portion configured for displacing vertically and for pitching about a pitch-axis member mediating between the pillar section and the upper-portion; and
c. electronic circuitry programmed to cause the upper portion to simultaneously displace vertically and pitch about the pitch-axis member to pivot the motor-control unit about a remote target point to align an operative vector of the motor-control unit with a target vector at the remote target point.

19. The positioning system of claim 18, wherein the aligning of the operative vector with the target vector includes:
i. lining up the operative vector of the motor-control unit to intercept the remote target point, and
ii. pivoting the motor-control unit about the remote target point to align the operative vector of the motor-control unit with a target vector at the remote target point.

20. The positioning system of either one of claim 18 or 19, wherein the aligning of the operative vector with the target vector includes aligning an offset vector with an offset target vector.

21. The positioning system of any one of claims 18 to 20, wherein the electronic circuitry is programmed to cause the upper portion to simultaneously displace vertically and pitch about the pitch-axis member while the motor-control unit is secured to the docking interface.

22. The positioning system of claim 21, wherein the electronic circuitry is programmed to cause the upper portion to simultaneously displace vertically and pitch about the pitch-axis member while a surgical arm is proximally seated in a surgical-arm-receiving volume of the motor-control unit.

23. The positioning system of either one of claim 21 or 22, wherein a surgical-arm-receiving volume of the motor-control unit is configured to receive a proximal portion of a surgical arm.

24. The positioning system of either one of claim 22 or 23, wherein a surgical arm proximally seated in the surgical-arm-receiving volume is aligned with the operative vector.

25. The positioning system of any one of claims 18 to 24, wherein the electronic circuitry is additionally programmed to cause the docking interface to displace longitudinally simultaneously with the vertical displacing and pitching of the upper portion.

26. The positioning system of any one of claims 18 to 25, wherein the electronic circuitry is additionally programmed to cause the docking interface to displace longitudinally after the aligning of the operative vector with the target vector.

27. The positioning system of any one of claims 18 to 26, additionally comprising a visual aid for aligning the operative vector with the target vector.

28. A positioning system for a robotic-surgery device, the positioning system comprising:
a. an upper portion comprising a docking interface adapted for securing thereto a motor-control unit of a robotic-surgery device; and
b. a lower portion comprising:
i. respective pluralities of support elements arranged to support the positioning system in respective positioning, position-adjusting, and stationary operating modes, the respective pluralities comprising respective pluralities of wheels, ball casters, and wheelless legs, and
ii. one or more mode-change pedals configured to transfer support of the positioning system from one respective plurality of support elements to another, the mode-change pedals arranged such that (A) an application of a mode-change pedal when the positioning system is supported by the plurality of wheels is effective to transfer support of the positioning system to the plurality of ball casters, and (B) an application of a mode-change pedal when the positioning system is supported by the plurality of ball casters is effective to transfer support of the positioning system to the plurality of wheelless legs.

29. The positioning system of claim 28, wherein at least one of the one or more mode-change pedals is arranged to lift the plurality of wheels when applied.

30. The positioning system of either one of claim 28 or 29, wherein the one or more mode-change pedals comprise a first mode-change pedal and a second mode-change pedal, and when the positioning system is supported by the plurality of wheels, an application of the first mode-change pedal is effective to transfer support of the positioning system to the plurality of ball casters, and an application of the second mode-change pedal is effective to transfer support of the positioning system to the plurality of wheelless legs.

31. The positioning system of any one of claims 28 to 30, wherein each wheel of the plurality of wheels has a first diameter and each ball caster of the plurality of ball casters has a second diameter, the first diameter being at least three times greater than the second diameter.

32. The positioning system of any one of claims 28 to 31, wherein the upper portion is configured for displacing vertically in response to a remote user input.

33. The positioning system of any one of claims 28 to 32, wherein the upper portion is configured for pitching, in response to a remote user input, about a pitch-axis member mediating between the lower portion and the upper-portion.

34. The positioning system of any one of claims 28 to 33, wherein the docking interface is configured for displacing longitudinally in response to a remote user input.

35. A method comprising:
a. providing a positioning system for a robotic-surgery device, the positioning system comprising (i) respective pluralities of support elements arranged to support the positioning system in respective positioning, position-adjusting, and stationary operating modes, the respective pluralities comprising respective pluralities of wheels, ball casters, and wheelless legs, and (ii) one or more mode-change pedals configured to transfer support of the positioning system from one respective plurality of support elements to another;
b. positioning the positioning system, with the positioning system supported by the plurality of wheels; and
c. applying one of the one or more mode-change pedals to transfer support of the positioning system to the plurality of wheelless legs, thereby placing the positioning system in the stationary operating mode.

36. The method of claim 35, additionally comprising, after the positioning of the positioning system:
i. applying one of the one or more mode-change pedals to transfer support of the positioning system from the plurality of wheels to the plurality of ball casters; and
ii. adjusting the positioning of the positioning system with the positioning system supported by the ball casters.

37. The method of either one of claim 35 or 36, wherein at least one of the one or more mode-change pedals is arranged to lift the plurality of wheels when applied.

38. Medical apparatus comprising:
a. a positioning system comprising a docking interface adapted for securing thereto a motor-control unit of a robotic-surgery, the motor-control unit comprising one or more surgical-arm-receiving volumes;
b. an illumination source coupled to the docking interface and operable to generate illumination in a positioning-system-distal direction, the illumination source being offset from the one or more surgical-arm-receiving volumes at least in a transverse direction that is orthogonal to the positioning-system-distal direction; and
c. an access-channel-assembly frame (i) comprising first and second iron sights aligned with each other to define an illumination target and an iron-sight-distal direction, and (ii) configured to have coupled thereto a surgical access channel shaped to allow traversal of respective distal portions of one or more surgical arms of the robotic-surgery device, the coupling being such that the first and second iron sights are offset from the coupled surgical access channel at least in a transverse direction that is orthogonal to the iron-sight-distal direction,
wherein a transverse-direction offset of the illumination source from the one or more arm-receiving volumes is substantially the same as a transverse-direction offset of the first and second iron sights from the access channel.

39. The medical apparatus of claim 38, wherein, when
i. the access-channel-assembly frame has a surgical access channel coupled thereto and is disposed at a surgical entry point and,
ii. the docking interface is positioned in a proximate-to-surgical-entry location in an assembled state in which the motor-control unit is mounted thereto, and one or more surgical arms are proximally received in respective arm-receiving volumes of the motor-control unit, and
iii. the illumination source is operated to generate illumination in the positioning-system-distal direction,
adjusting at least one of a position of the docking interface and an orientation of the docking interface, such that the illumination source illuminates the illumination target, is effective to align the one or more surgical arms for passage of distal ends thereof through the surgical access channel.

40. The medical apparatus of either one of claim 38 or 39, wherein the illumination source includes a laser illuminator.

41. The medical apparatus of any one of claims 38 to 40, wherein the iron sights are fold-out sights, and the defined illumination target and iron-sight-distal direction are defined with the sights folded-out.

42. The medical apparatus of any one of claims 38 to 41, wherein the docking interface is configured to be displaced vertically in response to a remote user input.

43. The medical apparatus of any one of claims 38 to 42, wherein the docking interface is configured to be pivoted, in response to a remote user input, about a pitch-axis member of the positioning system.

44. The medical apparatus of any one of claims 38 to 43, wherein the docking interface is configured for displacing longitudinally in response to a remote user input.

45. A method of employing the medical apparatus of any one of claims 38 to 44, the method comprising:
a. placing, at a surgical entry point, the surgical access channel, coupled to the access-channel-assembly frame;
b. positioning, in a proximate-to-surgical-entry location, the positioning system, in an assembled state in which the motor-control unit is mounted thereto, and the one or more surgical arms are proximally received in respective arm-receiving volumes of the motor-control unit;
c. operating the illumination source to generate illumination in the positioning-system-distal direction; and
d. adjusting at least one of a position of the docking interface and an orientation of the docking interface so that the illumination source illuminates the illumination target.

46. The method of claim 45, wherein the adjusting is effective to align the one or more surgical arms for passage of distal ends thereof through the surgical access channel.
